(19) United States
(12) Patent Application Publication
(10) Pub. No.: US 2024/0368511 A1
(43) Pub. Date: Nov. 7, 2024

(54) MICROFLUIDIC DEVICE FOR MANUFACTURING BIOSCAFFOLD AND USE THEREOF

(71) Applicant: BELEMENT INC., Ulsan (KR)

(72) Inventors: Joo Hun KANG, Ulsan (KR); Suhyun JUNG, Ulsan (KR)

(21) Appl. No.: 18/575,230

(22) PCT Filed: Jan. 25, 2023
(86) PCT No.: PCT/KR2023/001117

§ 371 Date: Jul. 24, 2024

(30) Foreign Application Priority Data
Jan. 24, 2022 (KR) 10-2022-0010222

(51) Int. Cl. C12M 3/06; C12M 1/00; C12M 1/42; C12M 3/00; C12N 5/071; C12N 5/077

(52) U.S. Cl. CPC C12M 23/16; C12M 21/08; C12M 23/38; C12M 35/04; C12N 5/0656; C12N 5/0658; C12N 5/069

(57) ABSTRACT

An aspect relates to a microfluidic device and use thereof. The microfluidic device of the present disclosure includes one or more micropillars therein, and thus, when the flow of blood is formed inside the device, shear stress is generated by the micropillars, leading to production of blood clots. The blood clots thus produced are vascularized and, when a wound site is treated therewith, simple wounds, viral infection caused by wounds, and chronic wounds can be significantly ameliorated. Blood vessels formed in the blood clots are aligned in the direction of blood flow so that a three-dimensional ECM structure suitable for tissues and organs can be manufactured.

### TECHNICAL FIELD

The present application claims priority to Korean Patent Application No. 10-2022-0010222, filed on Jan. 24, 2022, the disclosure of which is incorporated by reference in its entirety.

The present disclosure relates to a microfluidic device for manufacturing a bioscaffold and use thereof.

### Background Art

In the fields of regenerative medicine, drug delivery, and analysis, the development of biomaterial technologies needed to reproduce tissues and organs of patients is actively being carried out. Research on such development is being carried out with the aim of transplanting some or all of tissues and organs produced in vitro into patients to replace organs that have lost their function, or is aimed at developing patient-specific drug analysis technologies that can monitor the response of individual patients to drugs. Due to medical demands and rapid progress in the development of biomaterials, technologies of generating tissues and organs in vitro are rapidly developing, but it is still difficult to put them into practical use due to various technical problems.

The biggest problem among them is the technical limitation in reproducing extracellular matrix (ECM) structures of tissues and organs. ECM refers to components other than cells in tissue, and is a support that maintains the survival environment of cells and the shape of tissues. Therefore, development of appropriate ECM materials and manufacturing technologies is important to mimic the movement and growth signaling of cells in the actual tissues. Chemically synthesized materials and extracted natural materials have been tried as materials for manufacturing ECM, but recently, research on bio-derived ECM directly using animal or human cells or tissues is actively underway. As a representative example, materials left over from a decellularization process of removing cells from tissues of pigs or other animals are used for manufacturing ECM. However, since such a method utilizes xenogeneic tissue, there are a risk of acute immune response at the time of transplantation and a risk of long-term chronic response, and thus these risks needs to be explained. Therefore, to prevent rejection after transplantation, development of ECM using materials derived from a transplantee himself/herself is necessary.

Currently, various ECM manufacturing technologies are being studied to reproduce the three-dimensional ECM structure of tissues and organs. Electrospinning is the most widely used method today because it can mimic similarly actual fibers of ECM structurally. In particular, the electrospinning has the advantage that fibers can be patterned as aligning the fibers in a specific direction. In fact, the aligned fibers of ECM in the form of straight lines, grids, honeycomb patterns, etc. constitute tissues and organs, and such specific alignment of fibers of ECM is known to activate the functions of cells necessary for each organ, such as revascularization of vascular endothelial cells, differentiation of myofibers through alignment of muscle cells, implementation of neural circuits for nerve cells, and the like. However, to effectively handle a material solution used in the electrospinning, various conditions such as temperature, humidity, and airflow as well as viscosity, electrical properties, and ejection speed need to be controlled. In this regard, many expensive devices and environmental control systems are required, processing is difficult, and production reproducibility is poor, resulting in low yields of a manufactured product. In particular, unlike chemically synthesized materials, the aforementioned biomaterials derived from the actual tissues and organs cannot be finely controlled in viscosity, and thus application of the electrospinning thereto is more difficult. In such circumstances, there is a technical limitation in aligning bio-derived fibers of ECM with a diameter in hundreds of nanometers in various directions to manufacture a three-dimensional structure suitable for target tissues and organs.

### DISCLOSURE

### Technical Problem

One aspect provides a microfluidic device for manufacturing a bioscaffold, comprising: a substrate for supporting a biofluid containing extracellular matrix components and a bioscaffold produced by the fluid; and a cover positioned on top of the substrate and configured to induce gelation of the biofluid through a flow of the biofluid,
wherein the cover includes one or more inlets through which the biofluid is injected, a channel including one or more micropillars configured to induce shear stress to the injected biofluid, and one or more outlets capable of inducing a flow of the biofluid by discharging the biofluid from the channel.

Another aspect provides a method of manufacturing a bioscaffold, comprising injecting a biofluid containing extracellular matrix components into the microfluidic device for manufacturing a bioscaffold.

Another aspect provides a method of culturing cells by using a bioscaffold, comprising injecting a first biofluid containing extracellular matrix components and cells into the microfluidic device for manufacturing a bioscaffold.

Another aspect provides a composition for tissue transplantation, comprising the manufactured bioscaffold.

However, problems to be solved by the present disclosure are not limited to the aforementioned problems, and other problems not mentioned will be clearly understood by those skilled in the art from the descriptions below.

### Technical Solution

Provided is a microfluidic device for manufacturing a bioscaffold, comprising: a substrate for supporting a biofluid containing extracellular matrix components and a bioscaffold produced by the biofluid; and a cover positioned on top of the substrate and configured to induce gelation of the biofluid through a flow of the biofluid,
wherein the cover includes one or more inlets through which the biofluid is injected, a microfluidic channel configured to induce shear stress to the injected biofluid, and one or more outlets capable of inducing a flow of the biofluid by discharging the biofluid from the microfluidic channel.

The term “bioscaffold” as used in the present specification refers to a structure to which cells or other biological factors bind or support, and depending on the purpose of use, this term may be used interchangeably with “a material for tissue transplantation,” “a biocompatible support”, or “a support for cell culture”. The bioscaffold refers to a product generated from a biofluid having gelation or agglutination properties, and requires a high level of biomimicry. Such a high level of biomimicry is highly correlated with the activity or function of cells or other biological factors contained in the bioscaffold.

In an embodiment, the bioscaffold may contain a blood clot when the biofluid is blood. The term “blood clot” refers to a product generated through a blood coagulation process in vitro or in vivo, and may include an embolus. The blood clot may include an artificially generated blood clot and a blood clot that originally exists in blood. The blood clot may include blood cells (red blood cells, white blood cells, etc.), platelets, fibrin, calcium, von Willebrand factors, coagulation factors Xa, XIIIa, prothrombin, thrombin, fibrinogen, fibronectin, or neutrophil extracellular traps (NETs). Accordingly, in an embodiment, due to shear stress generated by a micropillar, the blood clot may be formed on the microfluidic device, such as at least a portion of the channel or at least a portion of the surface of the micropillar configured to induce shear stress to blood.

The term “microfluidic device for manufacturing a bioscaffold” as used in the present specification may be used interchangeably with “a device for manufacturing a material for tissue transplantation” or “a device for culturing cells within a bioscaffold,” depending on the purpose of use and processing a bioscaffold.

In an embodiment, the substrate for supporting a bioscaffold may be coupled to the cover. The substrate may have a larger cross-sectional area than the cover to support a biofluid injected to produce a bioscaffold and a bioscaffold formed by shear stress generated while the biofluid flows through the channel. The substrate may have a flat structure to generate an appropriate level of shear stress in the biofluid, but the structure is not limited thereto.

The substrate may further include one or more micropillars that can apply shear stress to the biofluid. In the case of the channel further including the micropillars, stronger shear stress can be applied to the biofluid, thereby facilitating the formation of a bioscaffold.

In addition, the substrate may have a structure configured to be easily separated from the cover for subsequent acquisition and culture of a scaffold, and to enable separation, may be temporarily coupled to the cover. The coupling method may be, but is not limited thereto, any method by which structures can be temporarily coupled together, such as by clamping.

In an embodiment, the cover may include an inlet through which the biofluid is injected, an outlet through which the biofluid is discharged, and a channel connecting the inlet with the outlet, thereby promoting gelation of the biofluid and providing a space to accommodate the formed bioscaffold. The cover may have a closed top, and the channel may be formed inside the cover.

The cover may be prepared by using polydimethylsiloxane (PDMS), polyethersulfone (PES), poly(3,4-ethylenedioxythiophene), poly(styrenesulfonate), polyimide, polyurethane, polyester, perfluoropolyether (PFPE), polycarbonate, or a combination of these polymers.

In an embodiment, the channel may have a tube-shaped structure connecting one or more inlets with one or more outlets. The channel may include one or more micropillars configured to induce shear stress to the biofluid, and the one or more micropillars may be patterned in the channel in an optimal shape for applying shear stress. The channel may be, but is not limited thereto, a portion having a rather large cross-sectional area connected to the one or more inlets and the one or more outlets.

The term “micropillar” as used in the present specification may protrude from the upper surface of the cover, and may be formed inside the cover when the substrate for supporting blood and a blood clot is coupled to the cover. The micropillar may protrude in the direction of the substrate positioned at the bottom of the cover.

In an embodiment, the micropillar may be configured to produce a bioscaffold depending on shear stress of the biofluid according to a change in the flow velocity of the biofluid that increases or decreases while the biofluid passes through the one or more micropillars. In detail, in such a structure, a bioscaffold may be produced from the flowing biofluid by increasing the surface shear speed of the flowing biofluid. In addition, the extracellular matrix components or other biological factors contained in the biofluid may be aligned and positioned according to the flow of the biofluid.

Without being limited to a specific theory, when the flowing biofluid flows into the microfluidic device through a pipe (e.g., an inlet) with a relatively large cross-sectional area, the flow velocity may accordingly increase due to the difference in cross-sectional areas, and the biofluid may undergo gelation or agglutination at room temperature due to the increased shear stress. In addition, when the bioscaffolds are formed around the one or more micropillars as described above, other scaffolds may be fixed and produced around the already formed scaffolds, thereby gradually narrowing gaps therebetween. In this regard, when the biofluid passes through one or more micropillars between the microscopic gaps thus formed, the shear speed may increase, thereby further promoting the production of scaffolds and alignment of the extracellular matrix components.

In an embodiment, the cross-section of each of the one or more micropillars may be n-polygonal or amorphous, and n may be 3 to 12. The cross-section, height, and interval of each of the one or more micropillars may be all the same or different from one another. The one or more micropillars may have any structure as long as a blood clot can be generated from the flowing blood by increasing the surface shear rate of the flowing blood, and for example, the structure may be a triangle, a diamond, a square, a pentagon, a hexagon, a heptagon, an octagon, an alphabet H, or a ribbon-like octagon in which at least two sides are recessed inwardly in a cross-section, or preferably, may be in the form of a diamond among squares.

In addition, the height or interval of the micropillar may be 0.1 μm to 5 cm, for example, 0.1 μm to 4 cm, 0.1 μm to 3 cm, 0.1 μm to 2 cm, 0.1 μm to 1 cm, 0.1 μm to 5 mm, 0.1 μm to 4 mm, 0.1 μm to 3 mm, 0.1 μm to 2 mm, 0.1 μm to 1 mm, 0.1 μm to 500 μm, 0.1 μm to 400 μm, 0.1 μm to 200 μm, 0.1 μm to 100 μm, 0.1 μm to 50 μm, 0.1 μm to 40 μm, 0.1 μm to 30 μm, 0.1 μm to 20 μm, 0.1 μm to 10 μm, 0.5 μm to 5 cm, 0.5 μm to 4 cm, 0.5 μm to 3 cm, 0.5 μm to 2 cm, 0.5 μm to 1 cm, 0.5 μm to 5 mm, 0.5 μm to 4 mm, 0.5 μm to 3 mm, 0.5 μm to 2 mm, 0.5 μm to 1 mm, 0.5 μm to 500 μm, 0.5 μm to 400 μm, 0.5 μm to 200 μm, 0.5 μm to 100 μm, 0.5 μm to 50 μm, 0.5 μm to 40 μm, 0.5 μm to 30 μm, 0.5 μm to 20 μm, or 0.5 μm to 10 μm.

The height of the micropillar may be the same as or lower than the height of the channel formed inside the cover. In the case where the height of the micropillar is the same as the height of the channel, the one or more micropillars may be in contact with the substrate for supporting blood and a blood clot when the substrate for supporting blood and a blood clot is coupled to the cover. The interval among the one or more micropillars may refer to a distance between one point on one micropillars and the same point on another micropillars. The interval among the one or more micropillars may be an interval through which one or two cells can pass.

The width (horizontal length) of the micropillar may be 0.1 μm to 5 cm, for example, 0.1 μm to 4 cm, 0.1 μm to 3 cm, 0.1 μm to 2 cm, 0.1 μm to 1 cm, 0.1 μm to 5 mm, 0.1 μm to 4 mm, 0.1 μm to 3 mm, 0.1 μm to 2 mm, 0.1 μm to 1 mm, 0.1 μm to 500 μm, 0.1 μm to 400 μm, 0.1 μm to 200 μm, 0.1 μm to 100 μm, 0.1 μm to 50 μm, 0.1 μm to 40 μm, 0.1 μm to 30 μm, 0.1 μm to 20 μm, 0.1 μm to 10 μm, 0.5 μm to 5 cm, 0.5 μm to 4 cm, 0.5 μm to 3 cm, 0.5 μm to 2 cm, 0.5 μm to 1 cm, 0.5 μm to 5 mm, 0.5 μm to 4 mm, 0.5 μm to 3 mm, 0.5 μm to 2 mm, 0.5 μm to 1 mm, 0.5 μm to 500 μm, 0.5 μm to 400 μm, 0.5 μm to 200 μm, 0.5 μm to 100 μm, 0.5 μm to 50 μm, 0.5 μm to 40 μm, 0.5 μm to 30 μm, 0.5 μm to 20 μm, or 0.5 μm to 10 μm, and preferably, 2 cm.

In addition, without being limited to a specific theory, at least one or a plurality of structures may cause a change in the flow velocity of the injected blood, and the larger the change, the higher the surface shear speed of the blood, resulting in the production of a blood clot. Such a surface shear speed of blood may be at the greatest in the plurality of structures, and a blood clot may be produced on the surface of the structures or close to the structures.

In an embodiment, the shear stress of blood capable of producing a blood clot from the flowing blood may be 0.01 dyne/cm² to 10,000 dyne/cm², or example, 0.01 dyne/cm² to 5,000 dyne/cm², 0.01 dyne/cm² to 2,500 dyne/cm², 0.01 dyne/cm² to 1,000 dyne/cm², 0.01 dyne/cm² to 500 dyne/cm², 0.01 dyne/cm² to 250 dyne/cm², 0.01 dyne/cm² to 100 dyne/cm², 0.01 dyne/cm² to 50 dyne/cm², 0.01 dyne/cm² to 25 dyne/cm², 0.01 dyne/cm² to 10 dyne/cm², 0.01 dyne/cm² to 5 dyne/cm², 0.01 dyne/cm² to 2.5 dyne/cm², 0.01 dyne/cm² to 1 dyne/cm², 0.01 dyne/cm² to 0.5 dyne/cm², 0.01 dyne/cm² to 0.25 dyne/dyne/cm², or 0.5 dyne/dyne/cm², or 0.01 dyne/dyne/cm² to 0.1 dyne/dyne/cm², but the shear stress is not limited thereto.

In an embodiment, the microfluidic device for manufacturing a bioscaffold may further include a frame that is seated on the cover and provides an internal space for guiding the second biofluid containing a functional factor to the gelled first biofluid. Here, the cross-sectional area of the frame providing an internal space may be wider than the cross-sectional area of the cover, but is not limited thereto.

In the present specification, the first biofluid may refer to a biofluid containing the aforementioned extracellular matrix components, and the second biofluid may refer to a fluid intended to regulate or promote the biological activity of the gelled bioscaffold and containing a functional factor. The functional factor may be a culture factor, a growth promoting factor, a differentiation inducing factor, or an expression inducing factor, and may refer to a protein, a cytokine, a conditioned medium, a virus, an extracellular vesicle, a cell, serum, RNA, an aptamer, PNA, and the like. However, application of the functional factor may be extended without limitation to any factor that can regulate or promote the biological activity. For example, the functional factor may include, but is not limited thereto, one or more selected from the group consisting of a fibroblast growth factor, a granulocyte colony-stimulating factor, interleukin-8, transforming growth factors alpha and beta, and a vascular endothelial growth factor.

In an embodiment, the first biofluid may be blood or a cell mixed with any other cells, and the second biofluid may be a fluid, a culture medium, or a differentiation medium, each containing a factor capable of promoting vascularization associated with plasma components.

The frame may be prepared by using polydimethylsiloxane, polyethersulfone, poly(3,4-ethylenedioxythiophene), poly(styrenesulfonate), polyimide, polyurethane, polyester, perfluoropolyether, polycarbonate, or a combination of these polymers.

In an embodiment, after being separated from the temporarily coupled substrate, a frame 30 may be coupled to the substrate and on top of the separated cover. The scaffolds formed on the cover may undergo gelation or agglutination, and thus may not leak out during and after coupling to the frame. In addition, when the frame providing the internal space is seated on the cover and is treated with the second biofluid, due to the presence of the internal space, the frame may be configured to guide the formation of a layer consisting of the second biofluid on the scaffold layer formed on the cover. In this regard, the second biofluid may be accommodated in the internal space of the frame while leakage of the second fluid to the outside is prevented.

According to an embodiment, the microfluidic device may align the extracellular matrix components and many cells present in the gelled bioscaffold in the direction of the microfluid flow, through the microfluid flow formed inside the channel and the shear stress generated thereby, thereby providing a bioscaffold or tissue-compatible transplant material having a high level of biomimicry.

Another aspect provides a method of manufacturing a bioscaffold, the method performed by a microfluidic device, which comprises: a substrate for supporting a bioscaffold; and a cover including one or more inlets, one or more outlets, and a channel connecting the one or more inlets with the one or more outlets, wherein the cover comprises one or more micropillars therein, and
comprising: injecting a biofluid containing extracellular matrix components into the microfluidic device; and forming a bioscaffold from the injected biofluid.

Since the method of manufacturing a bioscaffold includes or uses the same technical principles of the aforementioned microfluidic device for manufacturing a bioscaffold, the common contents between the two are omitted to avoid undue complexity of the present specification.

The term “biofluid” as used in the present specification refers to a biological fluid having gelation and agglutination characteristics, which may occur naturally or be prepared by artificial manipulation. In detail, the biofluid may be derived from a living body, or may be prepared by applying an injectable or administrable biocompatible material (fluid). Here, a biological sample known in the art may be applied without limitation. In addition, the biofluid may further contain extracellular matrix components or any other cells.

In an embodiment, the gelation or agglutination may be, but is not limited thereto, a phenomenon occurring by applying a physical or chemical stimulus, and may be induced by shear stress applied to the biofluid injected into the microfluidic device of an embodiment.

In an embodiment, the biofluid may be a body fluid (e.g., blood, plasma, serum, saliva, sputum, urine, lymph, cerebrospinal fluid, synovia, cystic fluid, ascites, interstitial fluid, or ocular fluid) isolated from mammals including humans, an extract from biological samples, or an artificial composite based on biocompatible materials.

Here, the term “blood” may refer to whole blood, artificial blood, pretreated blood (e.g., blood pretreated with an anticoagulant), some components of blood, such as plasma, plasma proteins, blood corpuscles, or blood cells, or lymph fluid, cerebrospinal fluid, or bone marrow, which may contain blood or some components of blood. In addition, the blood may be isolated blood from a subject, and that is, may refer to blood undergoing extracorporeal isolation from a subject or blood undergoing extracorporeal isolation and extracorporeal circulation.

The term “extracellular matrix components” as used in the present specification refers to components other than cells, and may play an important role in determining the function of cells as it includes various growth factors and cytokines secreted by cells. In an extracellular matrix environment similar to the environment created by cells, cells may best adapt to and be most physiologically active, and thus the cells may be used as scaffolds. Such extracellular matrix components may be extracellular matrix proteins, and may be aligned naturally or by an external stimulus.

The extracellular matrix proteins may be aligned according to the flow of the biofluid when the flow of the biofluid is induced by the microfluidic device of an embodiment. Although not limited thereto, the extracellular matrix proteins may include one or more selected from the group consisting of fibrin, collagen, fibronectin, von Willebrand factor, and laminins.

In an embodiment, the extracellular matrix proteins may be aligned according to the flow of the biofluid.

The term “aligned extracellular matrix proteins” as used in the present specification refers that the extracellular matrix proteins are aligned in the same direction as the flow direction of the microfluid. The scaffold formed by the microfluidic device of an embodiment may be an aligned array of extracellular matrix proteins, which is difficult to manufacture by a general method, and may be able to manufacture a biomimetic three-dimensional model (ECM platform) suitable for tissues and organs.

In an embodiment, the biofluid may further contain cells. The biofluid may further contain cells to form artificial tissues or organs through a subsequent culture process, wherein the cells may be of autologous origin for later use in transplantation or development of patient-specific drugs. The cells to be additionally contained may be, but are not limited thereto, one or more types of cells selected from the group consisting of vascular endothelial cells, muscle cells, stem cells, osteocytes, chondrocytes, cardiomyocytes, epidermal cells, fibroblasts, nerve cells, hepatocytes, enterocytes, gastric cells, skin cells, adipocytes, blood cells, immune cells, cell spheroids, and organoid cells.

In an embodiment, the cells may be vascular endothelial cells and myoblasts, which are muscle cells, and may undergo gelation or agglutination to form a scaffold as in the case of blood used as the biofluid. A scaffold formed by a biofluid containing the vascular endothelial cells may enhance expression of one or more selected from the group consisting of CD31, actin filament, and laminin, and may promote angiogenesis so that, at the time of transplantation, one or more histopathological features selected from the following may be induced at a transplantation site: (a) a decrease in expression of one or more selected from the group consisting of CD11b, IL6, IL-1β, CX3CR1, and TNFα; (b) promotion in angiogenesis; (c) migration of neutrophils to a transplantation site; (d) a decrease in ratios of neutrophils and M1 macrophages in the late stages of wound healing; and (e) an increase in ratios of M2 macrophages at the late stages of wound healing.

In an embodiment, the forming of a bioscaffold from the biofluid may refer to producing of a bioscaffold in the microfluidic device having the aforementioned structure according to the flow velocity of the injected biofluid. To this end, the flow velocity may be 0.01 mL/hour to 1,000 mL/hour, for example, 0.01 mL/hour to 100 mL/hour, 0.01 mL/hour to 10 mL/hour, 0.01 mL/hour to 1 mL/hour, 0.01 mL/hour to 0.1 mL/hour, 0.1 mL/hour to 1000 mL/hour, 0.1 mL/hour to 100 mL/hour, 0.1 mL/hour to 10 mL/hour, 0.1 mL/hour to 1 mL/hour, 1 mL/hour to 1000 mL/hour, 1 mL/hour to 100 mL/hour, or 1 mL/hour to 10 mL/hour, but may vary and be modified depending on types of a biofluid, contents of extracellular matrix components and cellular components, etc.

In an embodiment, the method of manufacturing a bioscaffold may further include treating the gelled biofluid or the formed bioscaffold with the second biofluid containing a functional factor, by using the frame providing an internal space. To this end, the aforementioned microfluidic device may further include the frame that provides an internal space, to be manufactured in the form of one set. Here, the first biofluid may refer to a biofluid containing the aforementioned extracellular matrix components, and the second biofluid may refer to a fluid intended to regulate or promote the biological activity of the gelled bioscaffold and containing a functional factor. The functional factor may be a culture factor, a growth promoting factor, a differentiation inducing factor, or an expression inducing factor, and may refer to a protein, a cytokine, a conditioned medium, a virus, an extracellular vesicle, a cell, serum, RNA, an aptamer, PNA, and the like. However, application of the functional factor may be extended without limitation to any factor that can regulate or promote the biological activity. For example, the functional factor may include, but is not limited thereto, one or more selected from the group consisting of a fibroblast growth factor, a granulocyte colony-stimulating factor, interleukin-8, transforming growth factors alpha and beta, and a vascular endothelial growth factor.

In the method of manufacturing a bioscaffold, matters regarding injection of a biofluid, mixing between a biofluid and cells, treatment of a functional factor, and culture conditions may be expanded and applied by employing techniques known in the art.

Another aspect provides a method of culturing cells by using a bioscaffold, the method performed by a microfluidic device, which comprises: a substrate for supporting a bioscaffold; a cover comprising one or more inlets, one or more outlets, and a channel connecting the one or more inlets with the one or more outlets; and a frame separately prepared and providing an internal space, wherein the cover comprises one or more micropillars therein, and

Comprising: injecting a first biofluid containing extracellular matrix components and cells into the microfluidic device; forming a bioscaffold from the injected biofluid; separating the substrate of the microfluidic device from the cover on which the bioscaffold is formed; coupling a frame that is seated on the separated cover and provides an internal space for guiding a second biofluid containing a functional factor to the gelled biofluid; and treating the frame seated on the cover with the second fluid containing a functional factor.

Since the method of culturing cells by using a bioscaffold includes or uses the same technical principles of the aforementioned microfluidic device for manufacturing a bioscaffold and the aforementioned method of manufacturing a bioscaffold, the common contents between the two are omitted to avoid undue complexity of the present specification.

In an embodiment, the method of culturing cells by using a bioscaffold may further include, after treating the frame with the second biofluid containing a functional factor, culturing the formed bioscaffold. Here, the resulting culture may provide a high level of biomimetic environment or an enhanced stimulus. Therefore, the method of culturing cells may effectively control the growth, differentiation, function, and biological activity of target cells.

According to an embodiment, a biofluid containing myoblasts may be used to perform the method of culturing cells in the manner described above. As a result, it is confirmed that myoblasts and myofibers within a blood clot are aligned according to the direction of the biofluid flow, and that myofiber differentiation markers are expressed at a high level, suggesting that the present disclosure may contribute to effective regulation of the growth, differentiation, function, and biological activity of cells.

In the method of culturing cells by using a bioscaffold, matters regarding injection of a biofluid, mixing between a biofluid and cells, treatment of a functional factor, and culture conditions may be extended by adopting the techniques known in the art.

Another aspect provides a composition for tissue transplantation, comprising a bioscaffold prepared by the aforementioned method of manufacturing a bioscaffold.

Since the composition for tissue transplantation includes or uses the same technical principles of the aforementioned microfluidic device for preparing a bioscaffold and the aforementioned method of preparing a bioscaffold, the common contents between the two are omitted to avoid undue complexity of the present specification.

The term “transplantation” as used in the present specification refers to a process of transferring viable tissues, cells, or artificial supports that accommodate the same, from a donor to a recipient with the intent of maintaining the functional integrity of the transplanted tissues or cells in the recipient.

The term “composition for tissue transplantation” as used in the present specification refers to a tissue-engineered construct intended to promote the recovery and regeneration of damaged tissues by attachment of biological cells or tissues, more specifically cells derived from damaged tissues, cells involved in the recovery of damaged tissues, or fragments of tissues differentiated therefrom. Here, the term “tissue attachment” as used herein refers to the direct or indirect adsorption of the composition to be transplanted to the matrix or other tissues while maintaining its intrinsic biological activity. In an embodiment, the composition for tissue transplantation may be for wound healing or recovery.

In an embodiment, a bioscaffold prepared by using the microfluidic device or according to the method of manufacturing a bioscaffold according to an embodiment, or materials for tissue transplantation may have a high level of biomimicry in terms vascularization and bundle formation of myofibers, and excellent biological activity, and thus the present disclosure may provide the composition for tissue transplantation having excellent tissue engineering characteristics.

### Advantageous Effects

According to a method of manufacturing a bioscaffold and a device for manufacturing a bioscaffold according to an aspect, extracellular matrix components and many cells present in a gelled bioscaffold may be aligned in the direction of the microfluid flow, through the microfluid flow and shear stress formed inside a channel, thereby manufacturing a bioscaffold or material for tissue transplantation with a high level of biomimicry.

According to a method of culturing cells by using a bioscaffold and a device a device for culturing cells according to an aspect, the growth, differentiation, function, biological activity of cells may be effectively regulated, and thus the present disclosure can be utilized in a wide variety of research fields.

### DESCRIPTION OF DRAWINGS

FIG. 1 is a view schematically illustrating a microfluidic device according to an embodiment.

FIG. 2 is a view schematically illustrating a microfluidic device according to an embodiment.

FIG. 3 is a perspective view illustrating the internal structure of the microfluidic device of FIG. 2.

FIG. 4 is an exploded perspective view illustrating the microfluidic device of FIG. 2.

FIG. 5 is a plan view illustrating a cover of FIG. 2.

FIG. 6 is an exploded perspective view illustrating a microfluidic device according to another embodiment.

FIG. 7A is a diagram showing a formation process of a blood clot in vivo and an operation method of a microfluidic device of an embodiment applying the formation process.

FIG. 7B is a diagram confirming shear stress generated by micropillars within a microfluidic device of an embodiment.

FIG. 7C is a diagram confirming vWF fibers formed around micropillars in a microfluidic device of an embodiment.

FIG. 7D shows photographs of blood clots formed over time when blood flows through a microfluidic device of an embodiment.

FIG. 7E is a graph quantitatively showing a level of blood clots formed after blood flows through a microfluidic device of an embodiment for 15 minutes.

FIG. 8A is a diagram showing a method of producing blood clots by using a microfluidic device of an embodiment, wherein (A) of FIG. 8A shows a microfluidic device consisting of an inlet, an upper part, a lower part, and an outlet; (B) of FIG. 8A shows injection of blood through the inlet; (C) of FIG. 8A shows formation of blood clots within the microfluidic device by the flow; and (D) of FIG. 8A shows separation of a cover and a substrate of the microfluidic device.

FIG. 8B is a diagram showing a method of producing blood clots by using a microfluidic device of an embodiment, wherein (A) of FIG. 8B shows seating of a PDMS frame on the edge of blood clots formed on a cover; (B) of FIG. 8B shows coating of the blood clots by pouring plasma mixed with fibroblasts over the blood clots; (C) of FIG. 8B shows separation of the blood clots after generating blood vessels within the blood clots by culturing for 4 days; and (D) of FIG. 8B shows the blood clots transplanted to a wound site.

FIG. 8C is a diagram showing a flow of blood when the blood flows through a microfluidic device of an embodiment.

FIG. 8D shows SEM images of formed blood clots when blood in a microfluidic device of an embodiment is under static conditions.

FIG. 8E shows SEM images of formed blood clots in blood when blood flows through a microfluidic device of an embodiment.

FIG. 8F shows images of blood clots formed in a microfluidic device of an embodiment, the blood clots being stained with CD41 and P-selectin.

FIG. 8G shows images confirming the activation level of platelets (P-selectin+) when blood flows through a microfluidic device of an embodiment.

FIG. 9A is a diagram confirming the formation of capillaries and alignment of fibers within blood clots, wherein FIG. 9A confirms the shape of fibers within blood clots under static conditions (Static) or blood clots induced by a flow of blood (Flow).

FIG. 9B shows images of the tubular structures of blood clots observed by staining actin filaments (F-actin), endothelial cells (CD31), and laminin, to confirm the level of capillaries formed within the blood clots.

FIG. 9C shows graphs confirming the expression levels of the observed actin filaments (F-actin), endothelial cells (CD31), and laminin, to confirm the level of capillaries formed within the blood clots.

FIG. 9D is a diagram showing the tubular structure of the blood clots confirmed through three-dimensional imaging of actin filaments (F-actin) and endothelial cells (CD31) (scale bar=50 μm), to confirm the level of capillaries formed within the blood clots.

FIG. 9E is a diagram showing the tubular structure of blood clots confirmed through three-dimensional imaging of laminin (scale bar=50 μm), to confirm the level of capillaries formed within the blood clots.

FIG. 10 is a diagram showing results confirming the number of white blood cells present in blood clots in which blood vessels form, wherein (A) of FIG. 10 shows electron microscopic images confirming the number of white blood cells in blood clots; (B) of FIG. 10 is a diagram confirming the number of neutrophilic granulocytes; and (C) of FIG. 10 is a diagram confirming the number of cells expressing CD45 and nuclei of the cells.

FIG. 11A shows images confirming the wound healing effects for 14 days for each type of blood clots and a control group, after an approximately 8 mm wound is created on the back of a mouse, to confirm the wound healing effects of blood clots in an animal model.

FIG. 11B is a diagram confirming the wound healing effects (wound closure levels) for 14 days and quantifying the same for each type of blood clots and a control group, after an approximately 8 mm wound is created on the back of a mouse, to confirm the wound healing effects of blood clots in an animal model.

FIG. 11C is a diagram confirming the epithelial aperture and collagen deposition levels, to confirm the wound healing effects of blood clots in an animal model.

FIG. 11D is a diagram observed by immunostaining for CD31 and α-SMA at the wound sites, to confirm the wound healing effects of blood clots in an animal model.

FIG. 11E is a diagram conforming the level of angiogenesis by quantifying the expression level of CD31, to confirm the wound healing effects of blood clots in an animal model.

FIG. 11F is a diagram confirming the level of vascular recovery in the recovery area by observing the expression levels of CD31 and α-SMA, to confirm the wound healing effects of blood clots in an animal model.

FIG. 12A shows images of a wound site (scale bar=1 cm) confirmed according to types of substance (NT, DFC, Static Vac, and Flow Vac) treated on Day 0, Day 5, Day 10, and Day 14, after blood clots are treated at the wound area, to confirm the wound healing effects of blood clots produced from an animal's own blood in a wounded animal model.

FIG. 12B shows graphs of the wound closure levels, collagen deposition, and skin thickness according to types of substance (NT, DFC, Static Vac, and Flow Vac) treated on Day 0, Day 5, Day 10, and Day 14, after blood clots are treated at the wound area, to confirm the wound healing effects of blood clots prepared from an animal's own blood in a wounded animal model.

FIG. 12C is a diagram of the wound site stained with H&E and Masson's trichrome (MT) 14 days after treatment with different substance (NT, DFC, Static Vac, and Flow Vac), to confirm the wound healing effects of blood clots when a wounded animal model is infected with bacteria.

FIG. 12D is a diagram confirming the wound closure levels after treatment with different substance (NT, DFC, Static VaC, and Flow VaC), to confirm the wound healing effects of blood clots when a wounded animal model is infected with bacteria.

FIG. 12E is a diagram confirming the epithelial gap and collagen deposition levels after treatment with different substance (NT, DFC, Static VaC, and Flow VaC), to confirm the wound healing effects of blood clots when a wounded animal model is infected with bacteria.

FIG. 12F is a diagram confirming confocal immunization images of blood vessels covered with perivascular cells on Day 5 and Day 14 after treatment with different substance (NT, DFC, Static VaC, and Flow VaC), to confirm the wound healing effects of blood clots when a wounded animal model is infected with bacteria.

FIG. 12G is a diagram confirming confocal immunization images of neutrophils in the wounded site on Day 5 and Day 14 after treatment with different substance (NT, DFC, Static VaC, and Flow Vac), to confirm the wound healing effects of blood clots when a wounded animal model is infected with bacteria.

FIG. 12H is a diagram confirming the number of neutrophils impregnated at the wounded area confirmed on Day 5 and Day 14 after treatment with different substance (NT, DFC, Static VaC, and Flow Vac), to confirm the wound healing effects of blood clots when a wounded animal model is infected with bacteria.

FIG. 12I is a diagram quantifying the density of formed blood vessels on Day 5 and Day 14 after treatment with different substance (NT, DFC, Static VaC, and Flow Vac), to confirm the wound healing effects of blood clots when a wounded animal model is infected with bacteria.

FIG. 12J is a diagram quantifying the level of neutrophils on Day 5 and Day 14 after treatment with different substance (NT, DFC, Static VaC, and Flow Vac), to confirm the wound healing effects of blood clots when a wounded animal model is infected with bacteria.

FIG. 13A is a diagram confirming and quantifying iNOS, a marker of M1 macrophages, in the healed wound by using confocal immunofluorescence, to confirm whether the wound in an animal model develops into a chronic wound.

FIG. 13B is a diagram confirming and quantifying CD163, a marker of M2 macrophages, in the healed wound by using confocal immunofluorescence, to confirm whether the wound in an animal model develops into a chronic wound.

FIG. 13C is a diagram confirming and quantifying the expression levels of CD11b, IL6, and IL-1β genes through PCR, to confirm whether the wound in an animal model develops into a chronic wound.

FIG. 13D is a diagram confirming and quantifying the expression levels of CXCR3 and TNF-α genes through PCR, to confirm whether the wound in an animal model develops into a chronic wound.

FIG. 14A is a diagram schematically showing a process of culturing a blood clot containing myoblasts.

FIG. 14B shows an image of myofibers within a cultured blood clot confirmed by using a scanning electron microscope.

FIG. 14C is a diagram confirming myofibers aligned according to the flow of blood in a microfluidic device.

FIG. 14D is a diagram confirming the expression levels of α-actin (sarcomeric) in myoblasts aligned according to the flow of blood in a microfluidic device.

### BEST MODE

Embodiments are subject to various modifications, and specific embodiments will be illustrated in the drawings and further described in the detailed description. The effects and characteristics of the embodiments, and methods of accomplishing the same, will be apparent by referring to the following detailed descriptions accompanied by the drawings. The embodiments may, however, be implemented in many different forms and should not be construed as limited to the embodiments set forth herein.

Hereinafter, the embodiments will be described in detail with reference to the accompanying drawings. The same or corresponding components will be denoted by the same reference numerals, and thus redundant description thereof will be omitted.

It will be understood that although the terms “first,” “second,” etc. may be used herein to describe various components, these components should not be limited by these terms. These components are only used to distinguish one component from another.

It will be further understood that the terms “comprises” and/or “comprising” used herein specify the presence of stated features or elements, but do not preclude the presence or addition of one or more other features or elements.

Sizes of elements in the drawings may be exaggerated for convenience of explanation. In other words, because sizes and thicknesses of components in the drawings are arbitrarily illustrated for convenience of explanation, the following embodiments are not limited thereto.

Hereinafter, the present disclosure will be described in detail with reference to Examples below. However, these Examples are for illustrative purposes only, and the scope of the present disclosure is not intended to be limited by these Examples.

As used in the present specification, a singular form may include a plural form, unless the context clearly indicates otherwise. In addition, the term “comprise, include” and/or “comprising, including” as used in the present specification is intended to specify the presence of the aforementioned figures, numbers, steps, operations, members, elements, and/or groups thereof, and does not exclude the presence or addition of one or more other figures, numbers, operations, members, elements. and/or groups.

In the present specification, the terms ‘top,’ ‘upper,’ bottom,' and ‘lower’ are relative concepts established from a point of an observer, such that, depending on the view of the observer, the ‘top’ may refer to ‘bottom,’ the ‘upper’ may refer to ‘upper,’ the ‘bottom’ may refer to ‘top,’ and the ‘lower’ may refer to ‘upper’.

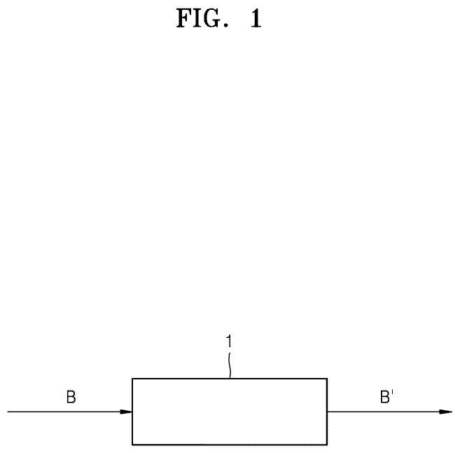

FIG. 1 is a diagram schematically illustrating a microfluidic device 1 according to an embodiment of the present disclosure.

Referring to FIG. 1, in the microfluidic device 1, when biofluid B containing extracellular matrix components and/or cells passes in one direction (B′), the biofluid may undergo gelation or agglutination inside the microfluidic device 1 to form a bioscaffold. Here, the extracellular matrix components or other cellular components contained in the biofluid B are aligned to have an orientation parallel to or equal to the moving direction B′ of the biofluid B, thereby simulating the microenvironmental structures of biological tissues such as blood vessels with greater reproducibility. In this regard, the bioscaffold itself formed inside the microfluidic device 1 may be applied as a material for tissue transplantation or as a culture capable of controlling the growth, differentiation, function, and biological activity of target cells.

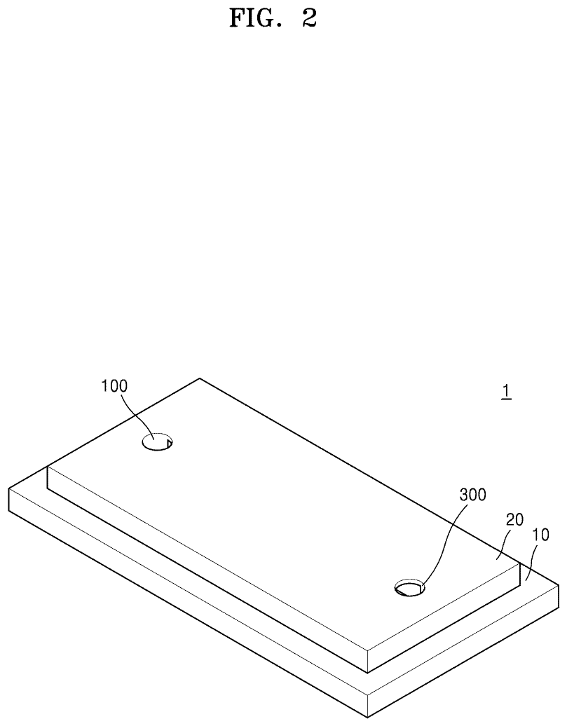

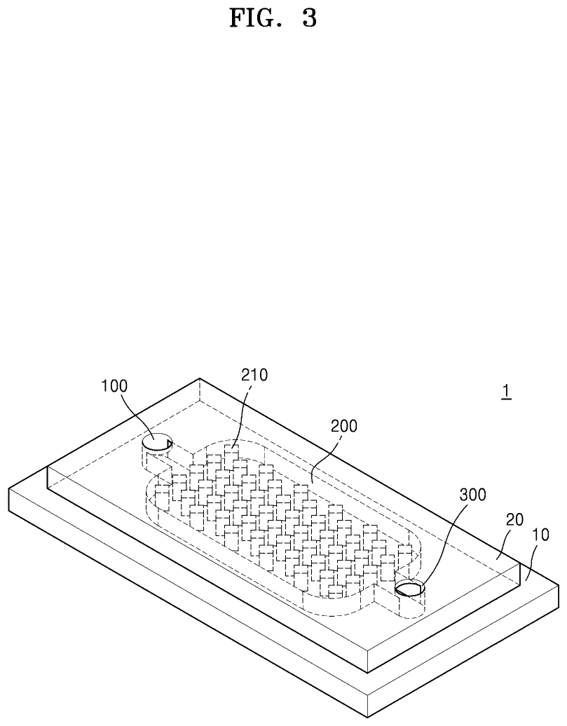

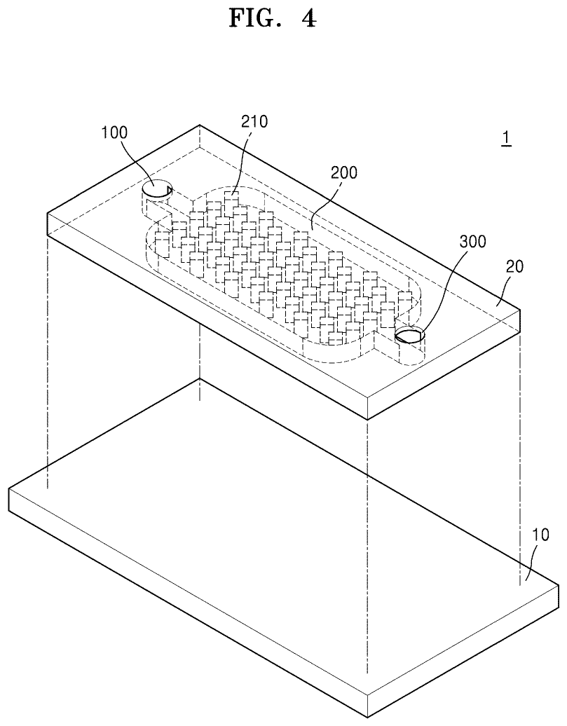

FIG. 2 is a perspective view illustrating the microfluidic device 1 according to an embodiment, FIG. 3 is a perspective view illustrating the inner structure of the microfluidic device 1 of FIG. 2, and FIG. 4 is an exploded perspective view illustrating the microfluidic device 1 of FIG. 2.

Referring to FIGS. 2 to 4, the microfluidic device 1 includes a substrate 10 and a cover 20 positioned on top of the substrate 10 and configured to induce gelation of the biofluid through the flow of the biofluid.

The substrate 10 may be connected, coupled, or tightly joined with the cover 20 to allow biofluid injected through an inlet 100 to move in a constant direction, and to serve as a support wall on which a bioscaffold may be formed by gelation or agglutination of the biofluid. In addition, the substrate 10, together with the cover 20, may provide a space where the bioscaffold may be arranged.

The cover 20 may promote gelation of the biofluid and provide a space to accommodate the formed bioscaffold. To this end, the cover 20 may have an inlet 100 through which the biofluid is injected, an outlet 300 through which the biofluid is discharged, and a channel 200 connecting the inlet 100 to the outlet 300. The cover 20 may have a closed top, and the channel 200 may be formed on the interior of the cover 20.

The channel 200 may be defined as a region that connects the inlet 100 to the outlet 300 and provides a passage through which the biofluid may move. The channel 200 may generate shear stress by contacting the biofluid.

In addition, the channel 200 may further include a plurality of micropillars 210. The channel 200 may provide a plurality of moving passages formed by the micropillars 210, and in this regard, may be defined as a space between the outer walls of the plurality of micropillars 210. The micropillars 210, as structures provided with the channel 200 which is a passage of the biofluid, may be in contact with the biofluid to generate shear stress. In this regard, the channel 200 provided with the micropillars 210 may apply stronger shear stress to the biofluid than a channel without micropillars. The micropillars 210 may be included in a patterned form in the channel 200 to generate shear stress at an effective level. The micropillars 210 are not limited thereto, but may be manufactured by using a lithography method. For example, the micropillars 210 may be manufactured by using a conventional soft-lithography method (Angew. Chem. Int. Ed. 1998, 37, 550-575) using poly(dimethylsiloxane) (PDMS) (Dow Chemical, USA). When the cover 20 is provided in the form of a closed top, the micropillars 210 may be provided in a form attached to the upper surface of the cover 20, and in this regard, may be provided to be in close contact with the upper surface of the substrate 10 that is arranged at the bottom of the cover 20. The micropillars 210 may further include a material that promotes gelation or agglutination, and the material may be surface-treated or applied on the micropillars 210.

In addition, in the microfluidic device 1 according to an embodiment, the substrate 10 and the cover 20 may be separated. Accordingly, the formed bioscaffold may be exposed to the outside in an area where the substrate 10 is separated, and the area exposed to the outside may be subjected to additional processing to modify or improve the characteristics of the bioscaffold or the cells within the bioscaffold. In addition, the formed bioscaffold may be obtained by removing the substrate 10 from the microfluidic device 1.

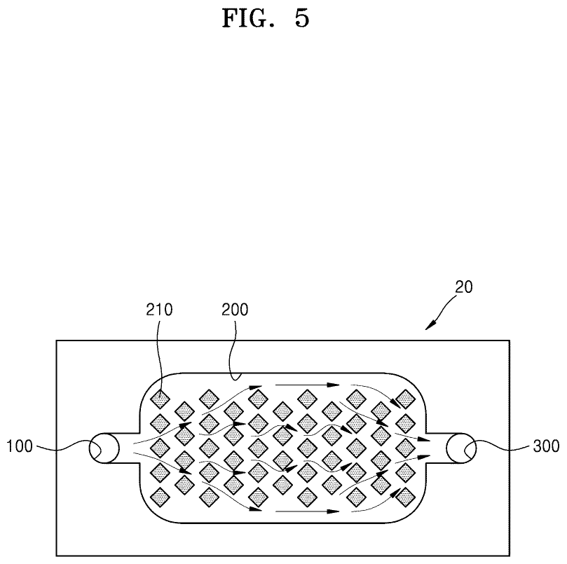

FIG. 5 is a plan view illustrating the cover 20 of FIG. 2, and is a view illustrating the internal structure of the cover 20 as view from the lower surface that is in contact with the substrate 10.

Referring to FIG. 5, as the biofluid injected through the inlet 100 arranged on one side of the cover 20 is discharged through the outlet 300 arranged on the other side of the cover 20, the biofluid within the channel 200 may have a constant flow velocity. Here, the plurality of micropillars 210 included in the channel 200 may increase or cause shear stress of the microfluid moving through the channel, to promote gelation of the microfluid, and the extracellular matrix components or other cellular components contained in the microfluid may be arranged to have an orientation parallel to or equal to the direction of movement of the biofluid.

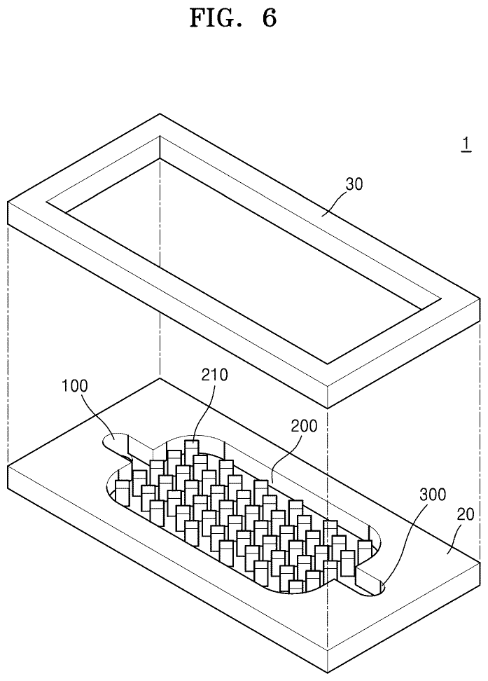

FIG. 6 is an exploded perspective view illustrating a microfluidic device 2 according to another embodiment.

Referring to FIG. 6, the microfluidic device 2 may further include a frame 30 that is seated on the cover 20 and configured to provide an internal space for guiding second biofluid containing a functional factor to the gelled biofluid.

The microfluidic device 2 may improve the characteristics of a bioscaffold formed on the cover 20, or may use the formed bioscaffold to improve the growth, differentiation, function, and biological activity of target cells included in the bioscaffold. In detail, the microfluidic device 2 may be implemented in the following manner. First, the cover 20 including the bioscaffold formed in the above-described manner and the substrate 10 are separated, and then, the cover 20 including the bioscaffold is inverted, so that the formed bioscaffold is positioned to be exposed to the outside, specifically, on the top. Next, the frame 30 may be arranged on the upper surface of the cover 20 where the bioscaffold is exposed, and the space within the frame 30 may be treated with the second biofluid containing a functional factor, and then cultured. Next, the cover 20 and the frame 30 may be each removed to obtain the bioscaffold or a cell culture. Here, the types of functional factors and specific culture methods may be associated with non-limiting factors known in the art, and may be appropriately changed depending on the intended use and types of target cells.

### EXPERIMENTAL EXAMPLES

### Experimental Example 1. Experimental Preparation and Protocols

### 1-1. Preparation of Mold to Manufacture Channel in Microfluidic Device

A mold to replicate a polydimethylsiloxane (PDMS) channel structure was manufactured by photolithography using an SU-8 photoresist, a method described in the existing literature. In detail, a PDMS prepolymer was mixed with a curing agent at a ratio of 10:1 ratio, and the mixture was poured into a silicon wafer mold. After curing the mold at 80° C. for 2 hours, a micropatterned PDMS was peeled off from the wafer. To allow liquid to pass through a channel, an inlet port and an outlet port were fabricated on the side of the PDMS slab. To improve the hydrophilicity of the PDMS channel, treatment with air plasma was performed thereon. To prevent trapping of air bubble, the PDMS channel was coated with a saline solution. When blood coagulated due to the micropatterned blood flow, glass was placed on the PDMS substrate and temporarily joined thereto by using a clamping method in consideration of disposal.

### 1-2. Formation of Blood Clots

Blood for experiments was collected from humans and rodents. Human blood was collected after receiving IRB approval (UNISTIRB-16-30G and UNISTIRB-19-23-C) from the Korea National Red Cross. Rodents were autologous transplant recipients, and mouse blood was collected via subclavian jugular vein and then stored in a blood collection tube containing heparin. A total of 4 mL of blood was collected over two days. Blood was recalcified with 5 μM of CaCl₂ before perfusion. A syringe pump controlled the rate of blood flow perfusing through the channel. Blood clots occurred gradually for 15 minutes, when the flow velocity of blood flow gradually decreased from 1 mL h⁻¹ to 0.1 mL h⁻¹. The glass on which the formation of blood clots was completed was separated from the PDMS substrate, and the blood clots were exposed to air. To coagulate autologous platelet rich plasma (PRP), PRP was extracted from blood according to the existing protocol, and then 5 μM PRP to which CaCl₂ was added was poured into a frame made of PDMS. Excess PRP was removed by scraping it off with a cover glass.

### 1-3. Image Confirmation by Scanning Electron Microscope

The obtained blood clots were washed with a saline solution, and then fixed with a 2.5% glutaraldehyde solution to prepare a sample. After washing the sample three times with a saline solution, the sample was dehydrated by successively treating it with 25%, 50%, 75%, 95%, and 100% ethanol solutions. After dehydration using a 1:1 mixed solution of hexamethyldisilane (HMDS) and ethanol, the sample was completely dried with 100% HMDS. The sample was stored in a vacuum desiccator until imaging. Before imaging with a scanning electron microscope, a gold-palladium thin film was deposited with a Hitachi sputter coater (20 mA. 60S).

### 1-4. Cell Culture

Human umbilical vein endothelial cells (HUVECs, Sartorius) and rat microvascular endothelial cells (RMVECs, CellBiologics) were grown in an endothelial cell medium (Sciencell). Normal human dermal fibroblasts (NHDFs, Lonza) and rat dermal fibroblasts (RDFs, CellBiologics) were cultured in FGM-2 medium (Lonza). All cells were subcultured 3 to 7 times at 37° C. and 5% CO₂ conditions until reaching 90% cell confluence.

### 1-5. Observation of Vascularization of Blood Clots

Endothelial cells (1×10⁵ cells/PBS 50 μL−1 in PBS) were mixed with 250 μL of blood recalcified with 5 μM CaCl₂. Blood was then injected into the blood coagulation device. The blood clots on micropost array substrate were removed with glass, and the red blood cells were immediately lysed with ACK lysis buffer upon exposure to air. After washing the blood clots with a culture medium, a PDMS frame, which had been soaked in 2 mg/mL⁻¹ polydopamine solution (Sigma) for 1 hour at room temperature to improve hydrophilicity, was placed on the substrate. 200 μL of lyophilized plasma containing dermal fibroblasts (1×10⁴ cells in PBS/20 μL⁻¹) was recalcified with 5 μm of CaCl₂, and then pipetted onto the blood clots in the PDMS frame. The formation of plasma gel was confirmed by culturing the cells at 37° C. for 15 minutes. Endothelial cells within the blood clots were matured in the microvascular network in EGM-2 MV medium (Lonza) for 3 days. It was confirmed that the microvascular blood clots could be gently removed and transferred from the micropost array substrate. The PDMS frame was cut with a blade for in vivo implantation.

### 1-6. Immunocytochemistry

The functionalized blood clots were fixed with 10% formalin, and then blocked with a 2% BSA buffer solution for 1 hour. After washing the cells with PBS, the cells were cultured with primary antibodies in blocking buffer overnight at 4° C. Primary antibody dilutions were prepared in 2% BSA buffer as follows: anti-CD41 (LSBIO) 1:100, anti-P-selectin (Novus Biologicals) 1:100, anti-CD31 (ThermoFisher Scientific) 1:100, anti-laminin (Abcam) 1:100, anti-collagen IV (Abcam) 1:100, anti-F-actin (ThermoFisher Scientific) 1:200, Fluor 555-conjugated anti-αSMA (ThermoFisher Scientific) 1:200.

After washing three times, the sample was cultured with secondary antibodies (goat anti-rabbit Alexa488 (ThermoFisher scientific), goat anti-rabbit Qdot647 (ThermoFisher scientific), goat anti-rabbit Qdot555 (ThermoFisher scientific), goat anti-mouse Alexa 594 (ThermoFisher scientific)) at room temperature for 2 hours in blocking buffer diluted at a concentration of 1:100. The nuclei of the blood clot samples were stained with Hoechst, and images were taken with an LSM 780 microscope (Zeiss). Then, immunofluorescence experiments were repeated for verification.

### 1-7. Western Blotting

Cell lysates were prepared by incubating Static Vac and Flow Vac in RIPA lysis buffer (Welgene) containing protease inhibitors (GenDEPOT) for 1 h at 4° C. The cell lysates were then centrifuged at 12,000×g, and the supernatant was collected. The protein evaluation of the lysates was performed by using a BCA protein assay kit (ThermoFisher Scientific). Laemmli sample buffer (Bio-Rad) was added to the lysate carrying the same amount of proteins, and the proteins were denatured by boiling at 100° C. for 10 minutes. The denatured protein sample was separated by electrophoresis on a 4% to 12% polyacrylamide gel (ThermoFisher Scientific), and then transferred to a PVDF membrane (0.2 μm, ThermoFisher Scientific). The membrane was washed three times for 15 min with TBST (10 mM Tris-HCl, 100 mM NaCl, and 0.1% Tween 20). The PVDF membrane was blocked in a TBST buffer for 2 hours at room temperature by using 4 wt % of dried milk (Sigma). The primary antibodies, laminin, collagen IV, CD31, and β-actin, were treated on the PVDF membrane overnight at the recommended concentration and at 4° C. Next, the membrane was cultured for 1 hour in a blocking solution containing horseradish peroxidase (HRP)-tagged secondary antibodies (Bioss) at room temperature. Proteins on the membrane were visualized with enhanced chemiluminescent HPR Substrate (SuperSignal West Pico PLUS Chemiluminescent Substrate, ThermoFisher Scientific), and images were obtained with a Chemiluminescence Imaging system.

### 1-8. Confirmation of Wound Healing Effect In Vivo

8-week-old male SCID nude mice and Sprague Dawley rats (ORIENT BIO Inc.) were used for animal experiments according to the guidelines of the Institutional Animal Care and Use Committee (IACUC) of the Ulsan National Institute of Science and Technology (UNIST). One day before surgery, the dorsal fur of the rat was shaved with depilatory cream, and anesthesia of the rat was induced with isoflurane and then continuously maintained with isoflurane. To control the pain experienced by the rats, tramadol (subcutaneous injection (20 mg/kg⁻¹)) was administered. Under sterile conditions, the dorsal skin was excised in a circular shape (diameter of 8 mm for mice and 14 mm for rats). A silicone ring (inner diameter of 10.82 mm for mice and 21.59 mm for rats) was placed on the wound site, and then the wound site was sutured to prevent natural skin contraction. The blood clots were placed in three layers on the wound site to be tested. During the experiments, a transparent dressing (e.g., Tegaderm) was used to prevent dehydration and scratching of the specimen. To prepare a bacterially infected wound model, methicillin-resistant Staphylococcus aureus (MRSA) was cultured in a lysogeny broth (LB) medium under aerobic conditions at 35° C. for 18 hours. MRSA (10⁸ CFU/40 μL PBS) was administered by pipetting into the wound site after 1 day. Photographs of the wound site were taken to confirm wound regeneration after 0, 5, 7, 10, and 14 days, and the size of the wound was expressed as a percentage (%) compared to the size on Day 0 by using an ImageJ software. After the experiments, the rodents were sacrificed on the 5th or 14th day, and the wound skin tissue was collected.

### 1-9. Histological Analysis

The wound tissue was collected from the healed wound site with a margin of about 2 mm. The collected sample was fixed in a 10% formalin solution overnight and embedded in paraffin. The tissue was cut to an appropriate thickness, and then immersed in a series of xylene solutions to remove paraffin. The sample was stained according to the standard procedures with hematoxylin and eosin (H&E) and trichrome of Masson, to confirm re-epithelialization and collagen deposition. For immunofluorescence staining, slides were deparaffinized and then rehydrated by using deionized water.

Antigen retrieval was performed in citrate buffer (10 mM sodium citrate, 0.01% Tween 20, pH6), blocking was performed with 2% BSA and 10% goat serum, and then tissues were cultured overnight with primary antibodies (Fluor 555- conjugated anti-αSMA (ThermoFisher Scientific) 1:200, anti-mouse/rat CD31 (R&amp;D Systems) 1:100, anti-human CD31 (Abcam) 1:100, anti-Neutrophil (LSBIO) 1:100, anti-CD163 (Abcam) 1:100, anti CD206 (Abcam) 1:100). The slide was cultured with fluorescently-conjugated antibodies for 2 hours. The nuclei were stained with Hoechst dyes, and images were obtained through a confocal laser microscope (LSM980, Zeiss). The fluorescence immunoassay was performed on the regenerated epithelium in a size of 5.1 mm×864 μm, and the intensity of expressed fluorescence was quantified by using the ImageJ software.

### 1-10. PCR Analysis

The regenerated wound tissue was collected by using an 8 mm biopsy punch, and then subjected to rapid freezing in liquid nitrogen. The frozen tissue was ground by using a mortar and pestle, and further homogenized with Trizol (Invitrogen) by using a handheld homogenizer. The aqueous phase of the Trizol extract containing RNA was purified using the RNeasy Mini Kit (Qiagen). The RNA was quantified by measuring absorbance at 260 nm by using Synergy Neo2 HTS Multi-Mode Microplate Reader (BioTek). cDNA was synthesized from 1,000 ng of total RNA according to the protocol of manufacturer of ReverTraAce qPCR RT Master Mix and gDNA remover kit (Toyobo).

The quantitative real-time PCR (qRT-PCR) analysis was performed by using a CFX connect real-time PCR detection system (Bio-Rad Laboratories) and SYBR green PCR master mix (Toyobo), and normal tissue without wounds was used. The gene expression levels were confirmed by performing normalization for GAPDH which is housekeeping gene.

### EXAMPLES

### Example 1. Manufacture of Microfluidic Device and Confirmation of Blood Clot Formation

In this embodiment, to confirm the efficacy of the microfluidic device in forming a bioscaffold according to an embodiment, blood containing various cells and extracellular matrix components was used as biofluid to determine whether a bioscaffold such as a blood clot was formed.

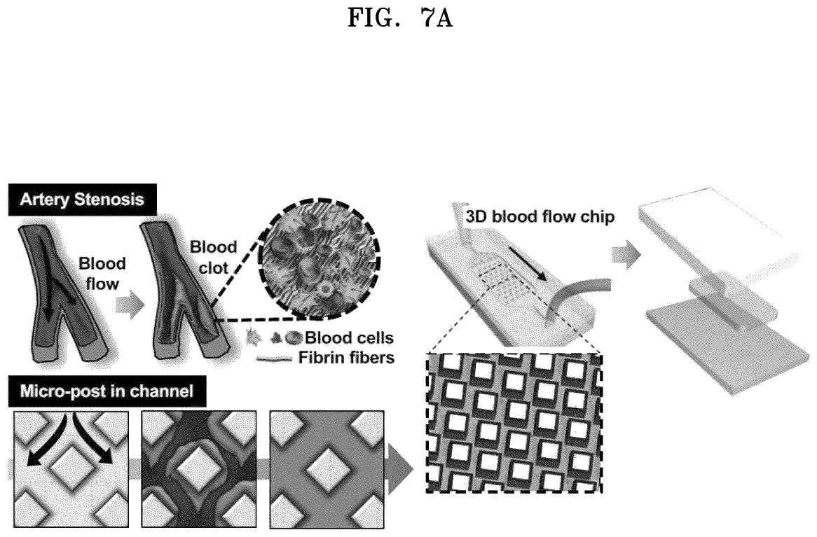

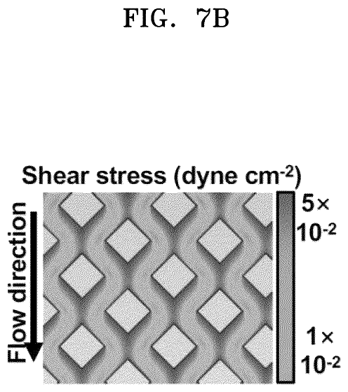

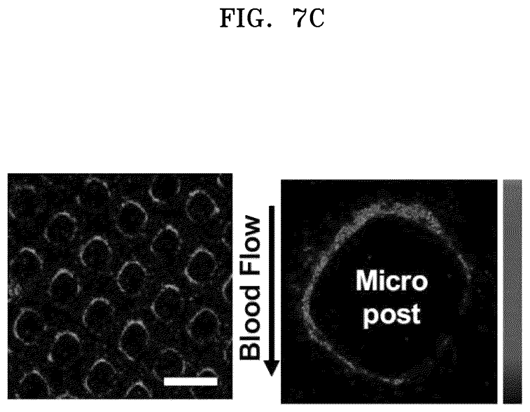

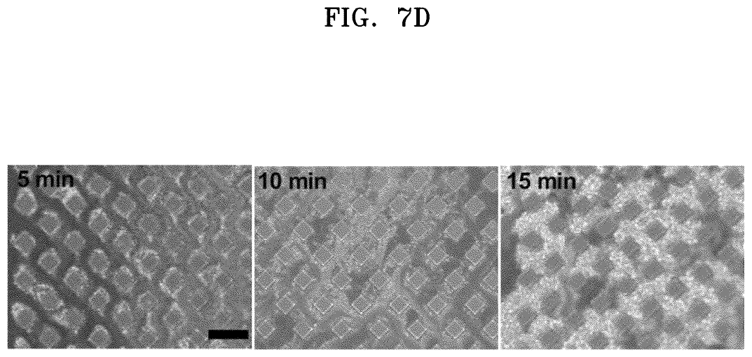

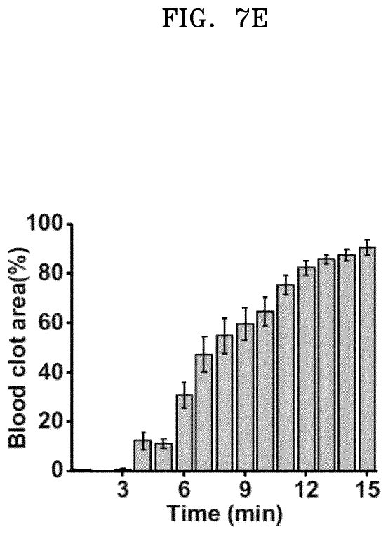

As described above, a microfluidic device with a structure that can utilize shear stress caused by micropillars was manufactured. When blood was injected into the microfluidic device, the blood flowing inside the channel of the microfluidic device caused high shear stress that aligned discontinuous fibrin and vWF fibers due to the small cross-sectional area of the microfluidic device or the micropillars arranged inside the microfluidic device, and through this, blood clots were designed in a manner similar to in vivo conditions (FIG. 7A). In detail, to generate uniform shear stress on the surface of the microfluidic channel, a structure was manufactured so that the micropillars in the channel come into contact with the blood. A computational fluid dynamic program (COMSOL v4.2, Comsol Inc., Burlington, MA, USA) was used to optimize the fluid flow conditions for blood coagulation, and the microfluidic device was equipped with diamond-shaped micropillars spaced at a distance of 100 μm (height×width×length: 50 μm×1.6 cm×2 cm). The shear stress around the micropillars was predicted to be in a range of 5×10⁻² dyne/cm², which is physiologically appropriate level for blood clots to form (>2×10⁻² dyne/cm²), at a flow velocity of 2 mL h⁻¹ (FIG. 7B).

After blood was injected into the microfluidic device manufactured by using the aforementioned method, the occurrence of blood clots was confirmed through time-lapse photography. Blood in an EDTA tube was recalcified with 5 μM CaCl. Through the time-lapse images, it was confirmed that blood coagulation began on the surface of the micropillar, where the greatest shear stress was expected, and that vWF fibers surrounded the micropillars, especially concentrated on the front part of the micropillars facing the blood (FIG. 7C). Blood clots accumulated in the micropillar area coated with vWF fibers, and it was confirmed that the blood clots filled the entire channel after about 15 minutes (FIGS. 7D and 7E).

### Example 2. Preparation of Blood Clot Using Biofluid Mixed With Cells and Evaluation of Characteristics Thereof

In this example, it was attempted to determine whether the characteristics of a bioscaffold could be changed by applying biofluid mixed with cells in a microfluidic device according to an example. To this end, a mixture of human umbilical vein endothelial cells (HUVECs), which is a type of endothelial cells, and blood was used as biofluid to confirm changes in the characteristics of blood clots.

### 2-1. Preparation of Blood Clots by Using Biofluid Mixed With Cells

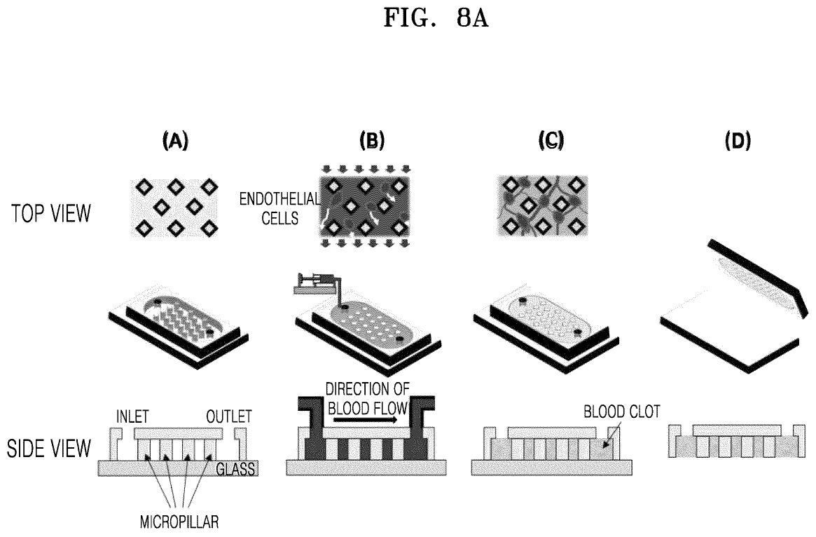

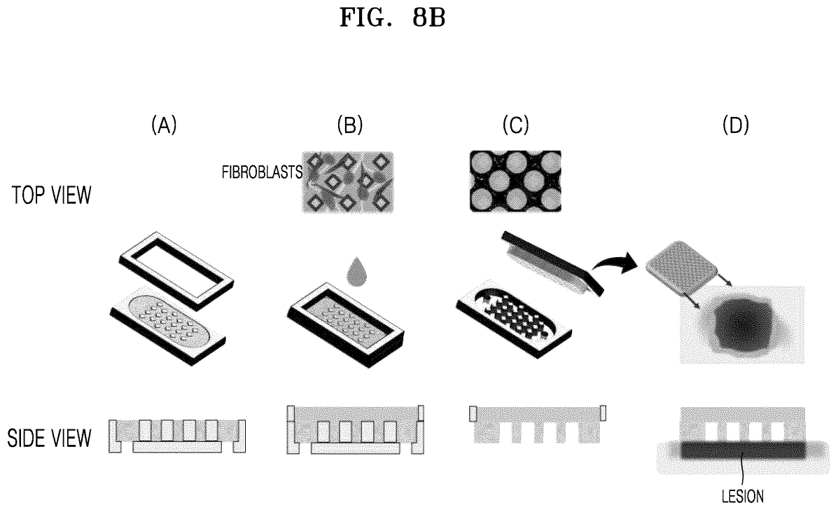

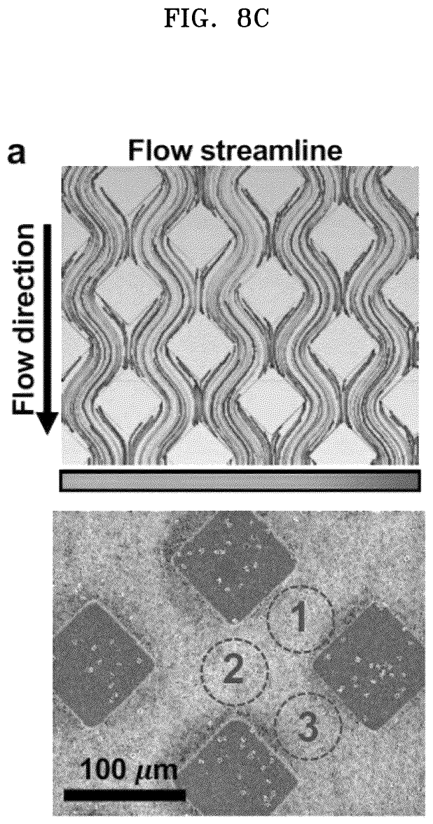

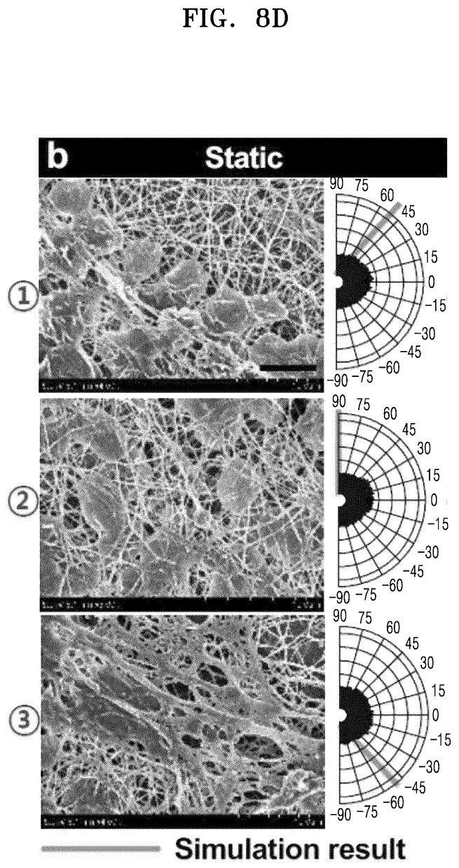

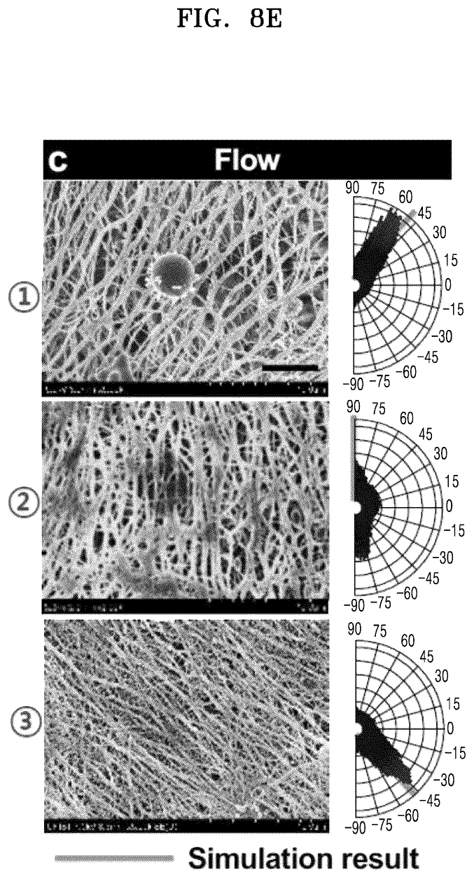

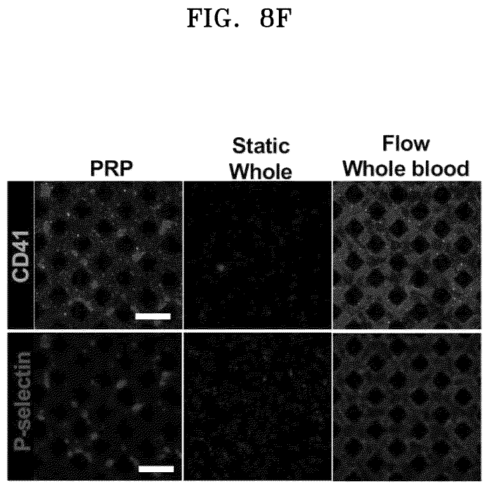

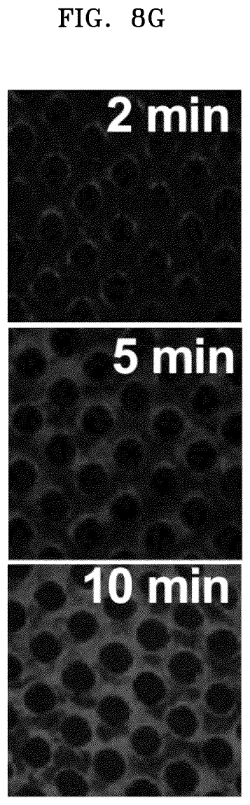

The preparation of blood clots by using a microfluidic device according to an embodiment was performed according to methods shown in FIGS. 8A and 8B. In detail, HUVECs, which is a type of endothelial cells, were mixed with human blood at a concentration of 1×10⁷ cells/mL. Blood was injected into the manufactured microfluidic device to form blood clots, and then the glass substrate positioned at the bottom was removed ((D) of FIG. 8A). Afterwards, the cover was inverted such that the formed blood clots were exposed on the top, and a frame made of PDMS material and having an area 1.2 times greater than the area of the formed blood clots and a height of 2 mm was placed on the cover made of PDMS material ((A) of FIG. 8B). Afterwards, 200 μL of fresh frozen plasma (Korea National Red Cross) in which 1×10⁶ cells/mL of human dermal fibroblasts (HDFs), which are skin fibroblasts, and 10 mM calcium chloride were mixed was added to the internal space of the frame to cover the entire upper part of the blood clots, and the plasma was solidified in a cell culture incubator at 36.5° C. for 20 minutes ((B) of FIG. 8B). The plasma was a blood component obtained by centrifugation at 5,000 g for 5 minutes at 4° C. within 6 hours of blood collection, and contained various blood coagulation factors. The solidified plasma fixed the blood clots during angiogenesis, and simultaneously strengthened mechanical characteristics to facilitate subsequent transplantation, and the skin fibroblasts mixed in the plasma may generate growth factors to help endothelial cells mature into blood vessels. Afterwards, a culture medium containing vascular endothelial growth factors was added or treated through the frame for 4 days, to form capillaries from the endothelial cells within the blood clots ((C) of FIG. 8B). The cover and frame that are made of PDMS material may each sequentially removed from the blood clots, and the blood clots were obtained ((D) of FIG. 8B).

### 2-2. Evaluation of Blood Clot Characteristics

Thrombogenic fibers in the blood clots were identified in the microfluidic device by using a scanning electron microscope. As confirmed in the simulation results, the image showed that the fibers were aligned along the blood flow line in the case of flowing blood, whereas the blood clots formed under static conditions showed an irregular fiber network (FIG. 8C). The fibers had the average diameter of 184.33±23.91 nm, and were confirmed to be similar to that of synthetic fibers extruded by electrospinning.

In addition, the activation level of platelet which is a factor responsible for angiogenesis, cell migration, and antibacterial activity in blood clots was confirmed. As a result of observing CD41, a surface membrane protein on platelets, by immunostaining, it was confirmed that blood clots formed by induced blood flow were had a greater number of platelets and evenly distributes platelets compared to blood clots that have coagulated under static conditions and platelet-rich plasma (PRP). It was also confirmed that the level of activated platelets determined by P-selectin increased more significantly in the blood clots formed by induced blood flow compared to blood clots formed under static conditions (FIG. 8D). The activation of platelets began 2 minutes after flowing whole blood through the device, and gradually increased until 10 minutes had elapsed (FIG. 8E).

Through the results above, when using the microfluidic device according to an embodiment, through mixing of biofluid and cells and a continuous supply of functional factors through the frame, it was confirmed that the bioscaffold was able to more closely mimic biological tissues or enhance biological characteristics thereof.

### Example 3. Characterization Evaluation According to Microfluidic Flow

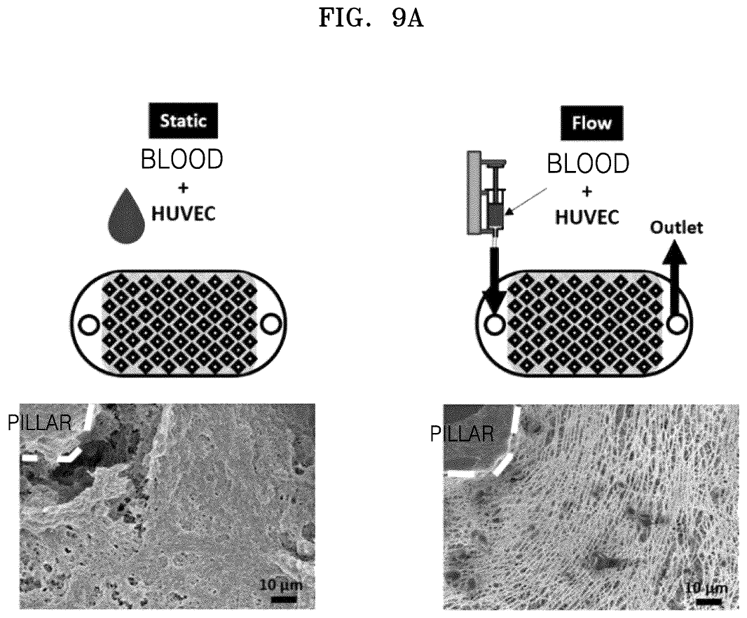

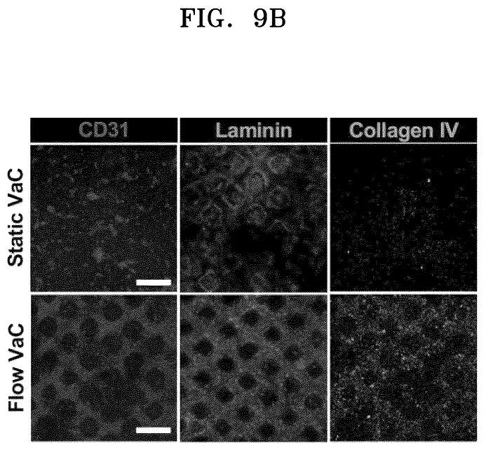

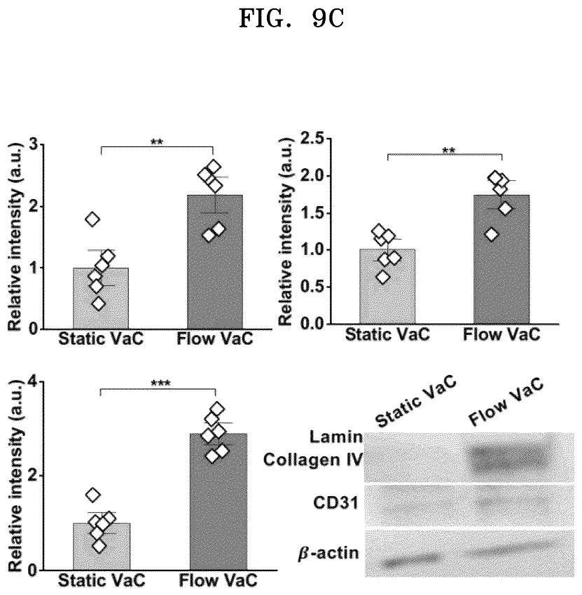

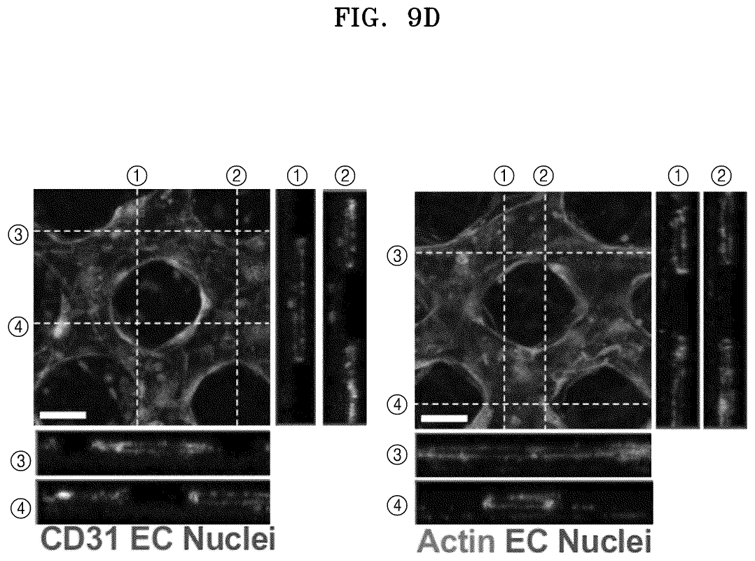

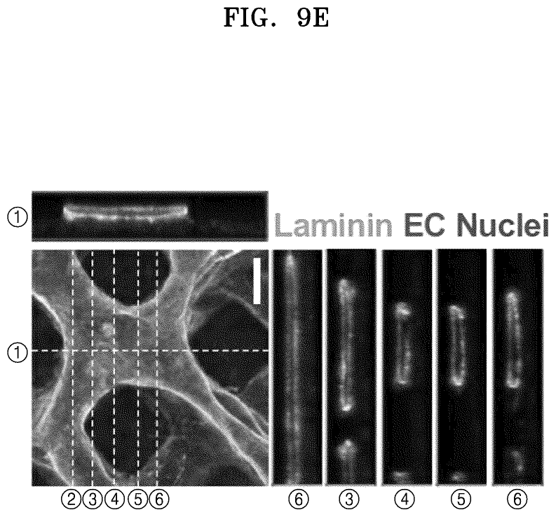

In this example, it was attempted to determine whether the flow of the biofluid was an important factor, along with the application of biofluid mixed with cells, in the microfluidic device according to an embodiment. In detail, a mixture of HUVECs and blood was used as biofluid in a microfluidic device, and blood clots (Static Vac and Flow Vac) were prepared in the same manner as described above in the absence of biofluidic flow (static) and in the presence of biofluidic flow (flow), respectively (FIG. 9A).

Afterwards, a comparative experiment was conducted on the Flow Vac and Static Vac. For this purpose, frozen-thawed plasma containing dermal fibroblasts (DFCs) was additionally added to enhance the angiogenic activity, and then the characteristics of the Flow Vac and Static Vac were compared. Endothelial cells were added to whole blood before coagulation began, so that the spiked endothelial cells became trapped in the blood clots. When blood clots were formed, the substrate at the bottom was removed. Afterwards, the DFCs were pipetted into the previously prepared frozen-thawed plasma, and positioned to expose the blood clots formed on the cover, in the same manner as in the examples above. Then, the plasma was treated thereon through the frame. The blood clots were cultured for 3 days, and consequently, had a honeycomb-shaped structure. In this regard, it was confirmed that the microvascular network was densely organized with fibers (bottom of FIG. 9A).

To confirm the function of the formed microvessels, characteristic marker proteins of mature blood vessels were identified. First, by using immunofluorescence, the expression levels of CD31, laminin, and collagen IV and the basement membrane deposition by endothelial cells were compared. As a result, it was confirmed that the level of CD31 in the Flow Vac was higher than that in the Static Vac, referring that the blood vessels formed by the Flow Vac are more mature (FIG. 9B). In addition, the levels of laminin and collagen IV were also found to be approximately 2 to 3 times higher in the Flow Vac (FIG. 9C). Moreover, the cross-sectional images of blood vessels stained for actin filaments and basement membrane proteins showed that blood vessels surrounded by endothelial cells in the Flow Vac formed a continuous and hollow lumen (FIGS. 9D and 9E). Through the results above, it was confirmed that the Flow Vac induced by the microfluidic device improved the formation of microvessels and physiological functions compared to the Static Vac.

### Example 4. Confirmation of Wound Healing Ability of Blood Clots Prepared by Microfluidic Device

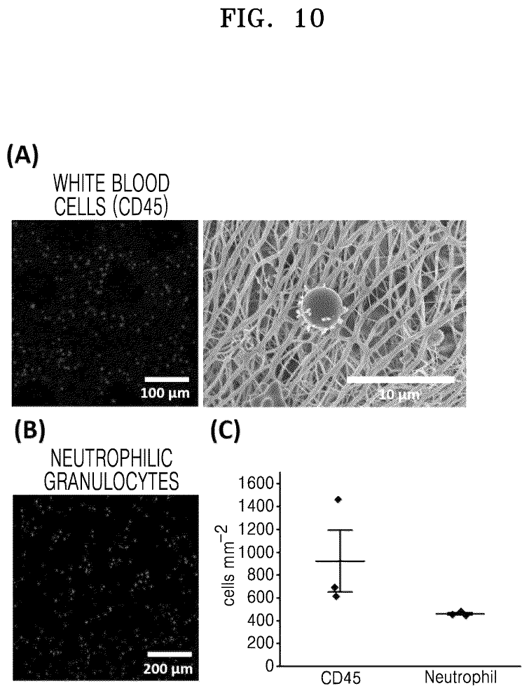

To confirm the wound healing ability of the blood clots prepared by the blood flow in the microfluidic device of an embodiment, the level of immune cells in the blood clots was confirmed through CD45 expression and scanning electron microscopy. As a result, it was confirmed that a large number of leukocytes were present in the blood clots (FIG. 10). The presence of autoimmune cells in these blood clots is expected to reduce the risk of abnormal immune responses against transplantation and the risk of infection, and is also expected to promote the activity of other immune cells in the skin that play a major role in wound recovery.

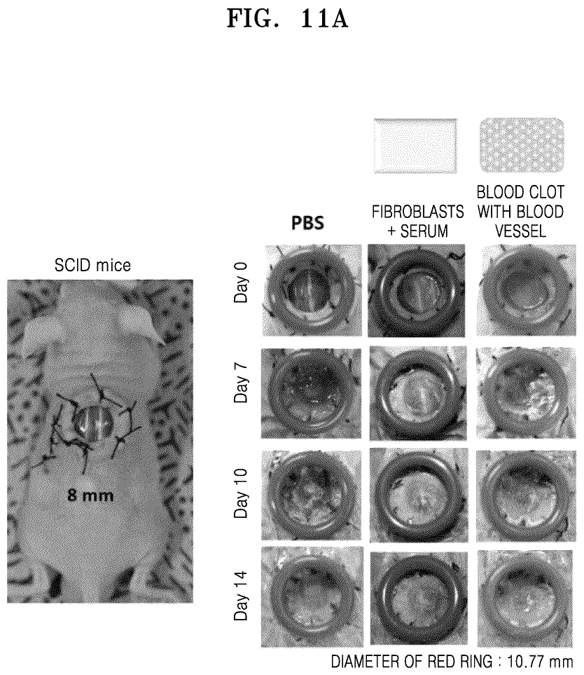

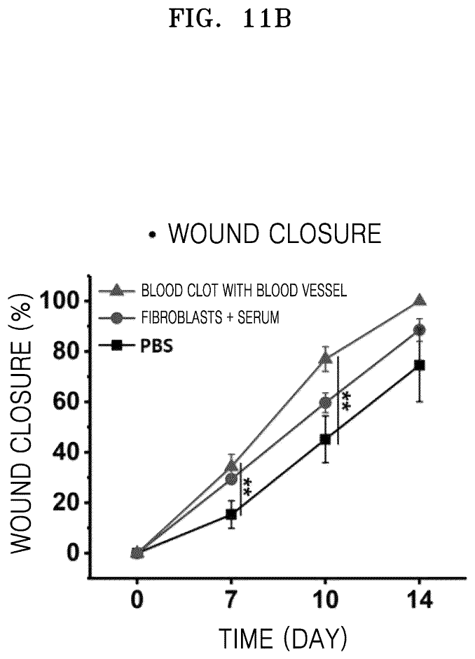

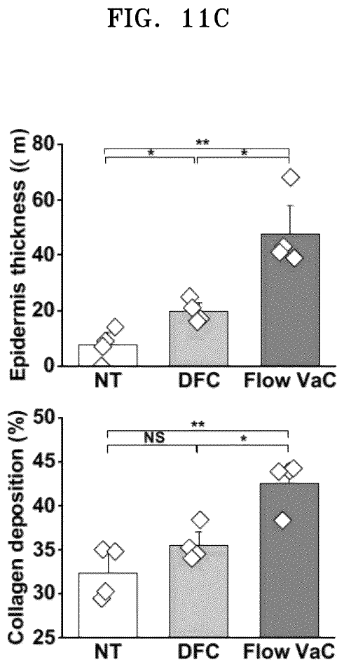

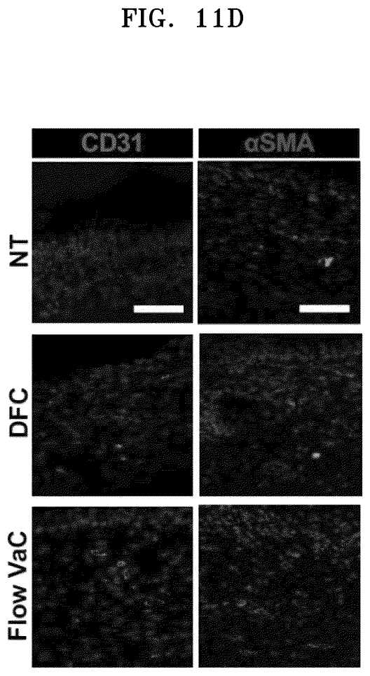

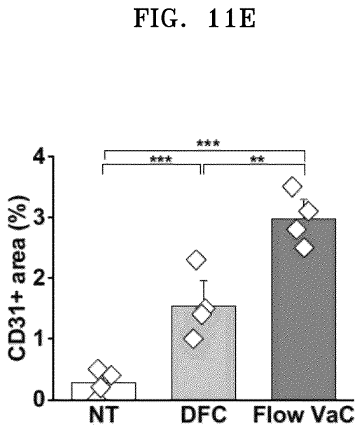

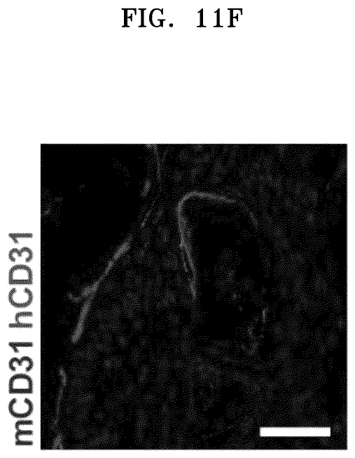

In addition, to confirm the specific wound healing ability of the blood clots, the level of pre-vascularization was confirmed by using DFCs as a control group. An 8 mm-thick wound was created on the back of an SCID mouse, and the wound site was treated with DFCs (hereinafter referred to as a DFC-treated group) or with blood clots in which the angiogenesis was induced (referred to as Flow VaC). In addition, as a control group, a group treated only with a cell medium was used (hereinafter referred to as an NT group). To confirm the treatment of the wound site, images showing changes in each group were photographed. After 10 days, the group treated with the blood clots in which the angiogenesis was induced showed a significant wound healing effect compared to the DFC-treated group and the NT group. By epidermis thickness measurement and Masson-Trichrome staining, the levels of wound re-epithelialization and collagen deposition were confirmed. As a result, after 14 days, the complete re-epithelialization occurred in the group treated with the blood clots in which the angiogenesis was induced, whereas the wound was still not completely healed in the control group (FIGS. 11A and 11B). Moreover, in the case of the blood clots in which the angiogenesis was induced, the expression level of collagen was significantly high, confirming that the wound could be regenerated without leaving a scar (FIG. 11C).

Next, 14 days after the surgery and collection of skin tissues, immunostaining targeting CD31 and αSMA antibodies, known as the most common markers of endothelial cells and alpha smooth muscle cells, was performed (FIG. 11D). As a result, regarding the CD31 positive area, it was confirmed that the group treated with the induced blood clots had 2 to 4 times as many positive areas as the control group (FIG. 11E). In addition, the immunostaining results show that the binding sites of human and mouse CD31 antibodies overlap (FIG. 11F), showing that the blood clots in which angiogenesis was induced were able to induce not only vascularization but also anastomoses of blood vessels. In other words, the blood clot induced by the microfluidic device showed an excellent wound healing effect.

### Example 5. Confirmation of Wound Healing Ability Against Bacterial Infection by Using Autologous Blood Clots

The wound healing effect on Sprague-Dawley rat was confirmed by using blood clots prepared by using mouse blood. In detail, experiments were performed on a group treated with blood clots generated from plasma containing dermal fibroblasts (referred to as DFC) and groups (referred to as Static VaC and Flow Vac) treated with blood clots in which vascularization was induced by using the microfluidic device according to an embodiment, respectively under conditions without biofluid flow (referred to as Static) and with biofluid flow (referred Flow). As a control group, a group treated with a cell medium (referred to as an NT group) was used.

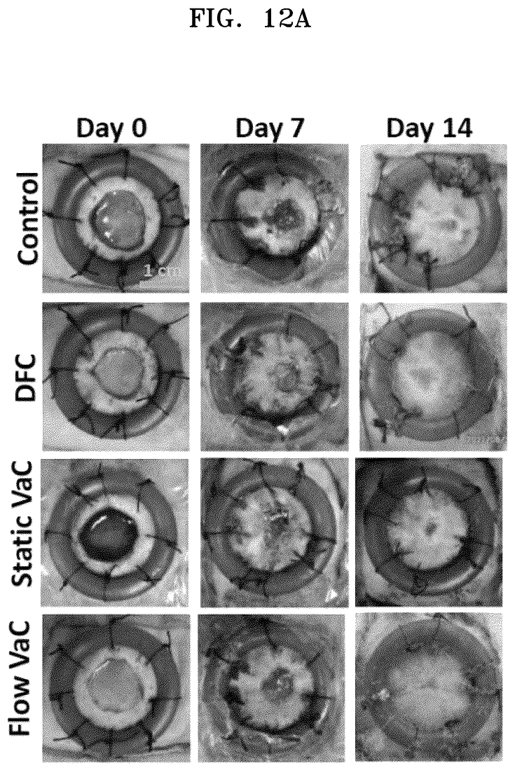

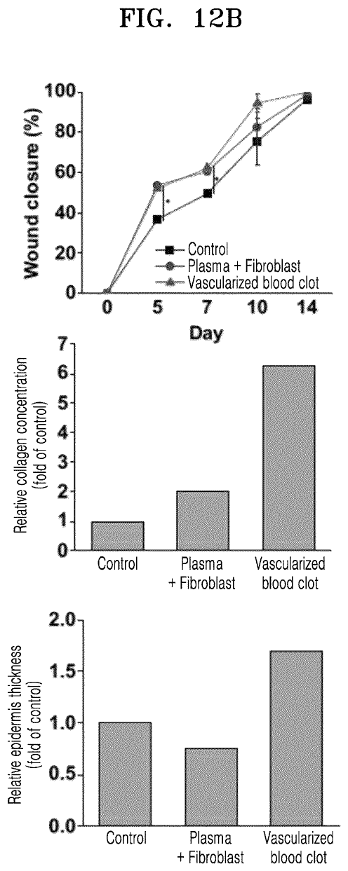

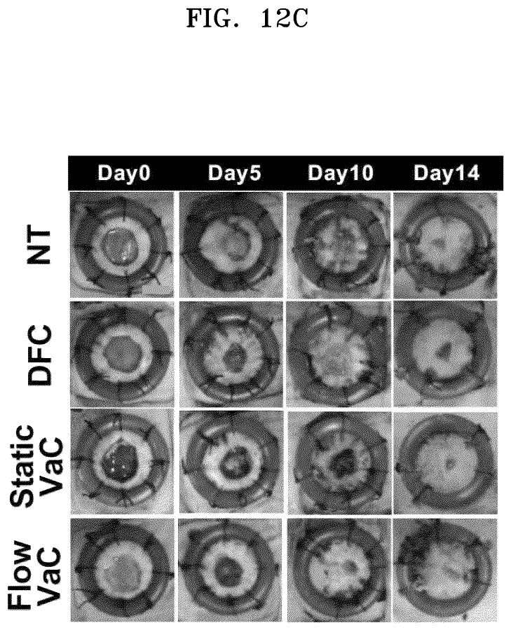

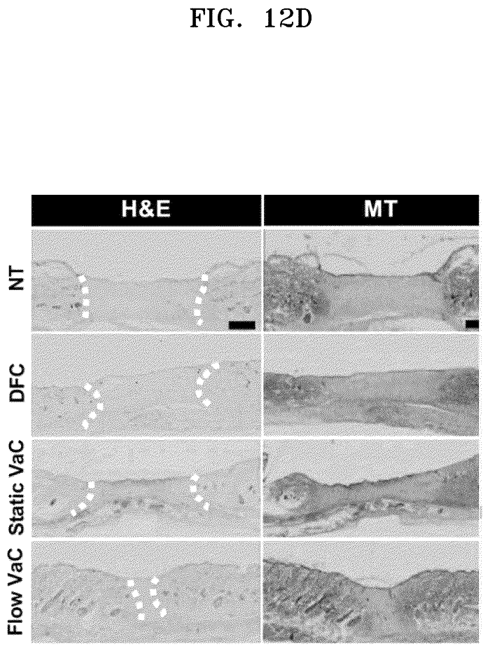

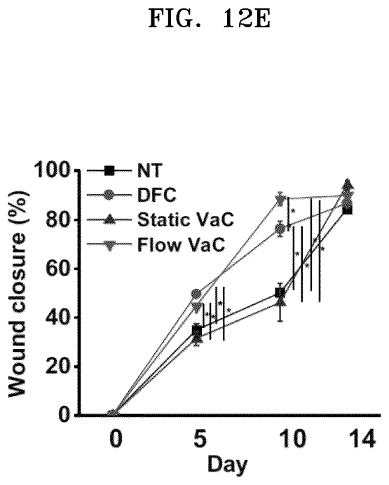

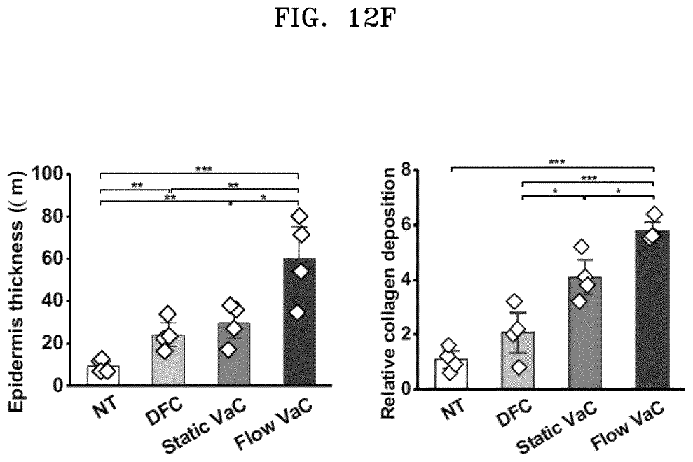

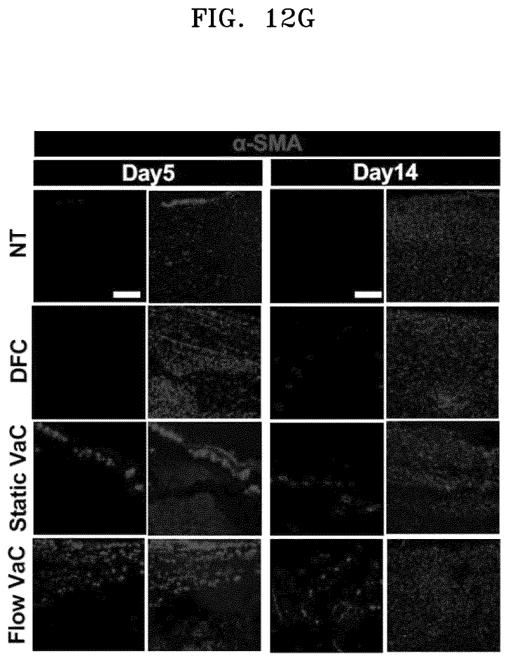

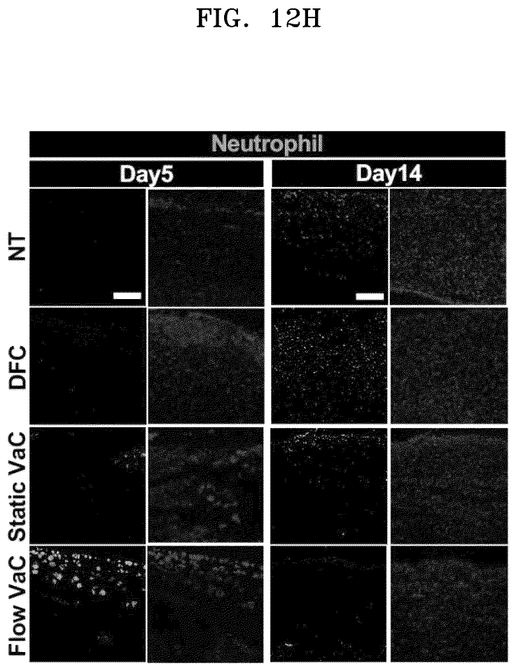

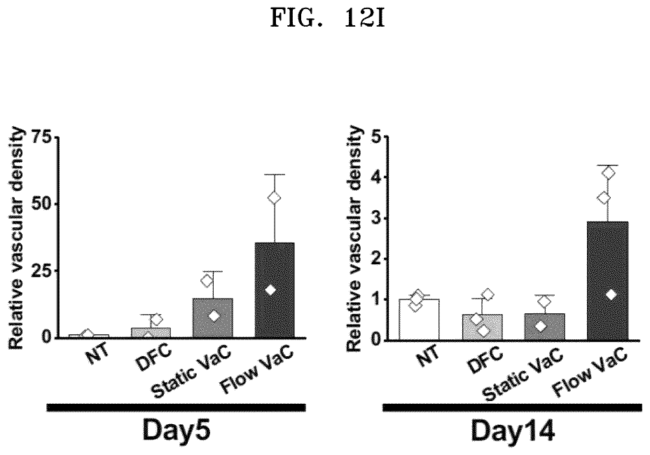

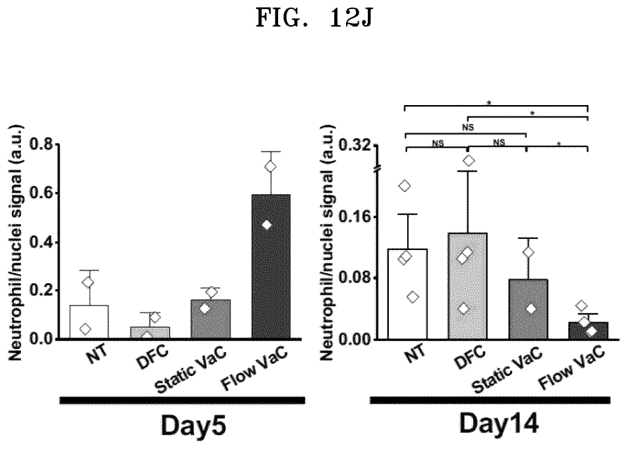

When autologous blood clots were transplanted to the wound site, the Flow VaC group showed a superior wound healing effect to other experimental groups (FIGS. 12A and 12B). In detail, the Flow Vac group showed a more significant effect than other groups in terms of epidermal thickness, collagen density, and microvascular density.

To determine whether the autologous blood clots could be used to treat sites where bacterial infection has progressed during the wound treatment as well as the simple wounds described above, experiments were carried out by using a biological model infected with methicillin-resistant Staphylococcus aureus (MRSA). One day after perforation of about 14 mm-diameter skin by biopsy, MRSA (1×10⁸ CFU) was inoculated into the wound site, and the infected wound site was photographed every 5 days (FIG. 12C). The group using DFCs (DFC) and the group treated with the blood clots induced by the flow (Flow VaC) showed the wound closure levels of about 49.77±1.32% and about 44.67±0.82%, respectively, on Day 5, showing therapeutic effects at a significant level (FIG. 12C). On the other hand, the Static VaC group showed the wound closure level of about 31.51±2.94%, and the control group showed the wound closure level of about 34.82±2.77%. The Flow VaC group showed the wound closure level of about 88.63±2.54% on Day 10, confirming that a very significant wound healing effect was exhibited. On Day 14, unlike other groups, it was confirmed that the Flow Vac group had undamaged epithelial structures and collagen that was neatly aligned (FIGS. 12D and 12F). In the DFC group and the control group, pus continued to form at the wound site and remained in the early stages of the wound healing process, and in the Static VaC group, pus was not observed at the wound site, showing a low level of the wound closure (FIG. 12E).

### Example 6. Confirmation of Inhibitory Effect on Development to Chronic Wounds

Among the wound healing effects on Sprague-Dawley rat confirmed by using blood clots prepared by using mouse blood, the inhibitory effect on development to chronic wounds was to be confirmed. In this example, the experimental groups and the control group were set up the same as in Example 5.

### 6-1. Histopathological Evaluation of Wound Site

An experiment to confirm the initial response to wound healing was carried out. As a result of performing αSMA staining, it was confirmed that capillaries were produced with 35 times higher density in the Flow Vac-treated group, whereas capillaries were produced with 3.5 times higher density and 14.5 times higher density, respectively, in the DFC-treated group or the Static Vac-treated group (FIGS. 12G and 12I). In addition, the number of neutrophils was counted and similar to the results described above. That is, a large amount of neutrophils were observed in the Flow Vac-treated group at the beginning of wound healing, on Day 5, and by Day 14, neutrophils were present at very low levels (FIGS. 12H and 12J). In addition, it was confirmed that neutrophils identified in the Flow VaC-treated group significantly overlapped with the positions of αSMA-stained capillaries. Considering that the number of neutrophils decreased in the late stage of wound healing, it was confirmed that the wounds in the Flow Vac-treated group did not proceed to chronic wounds.

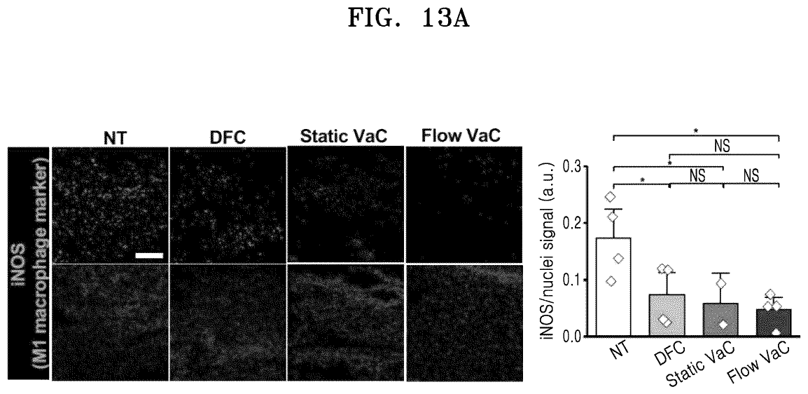

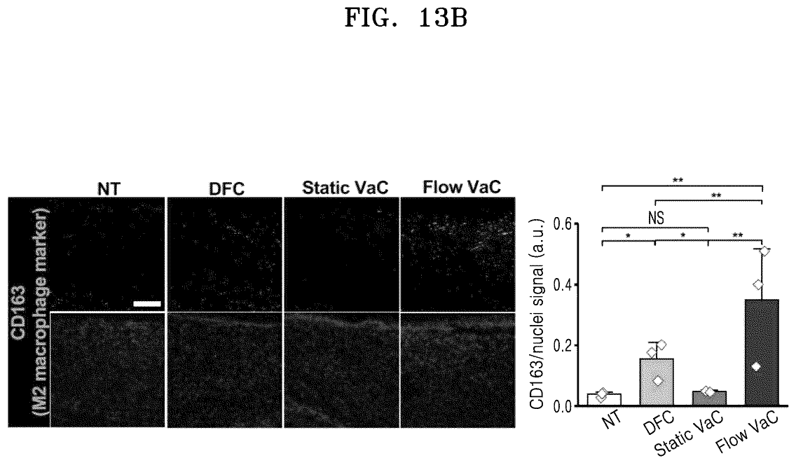

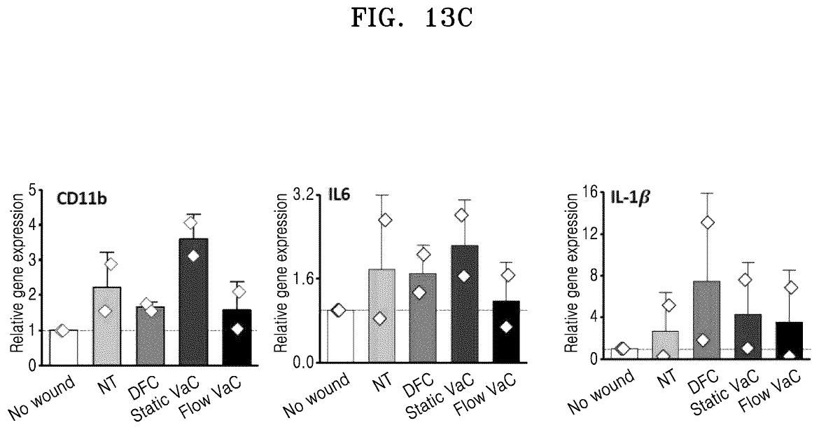

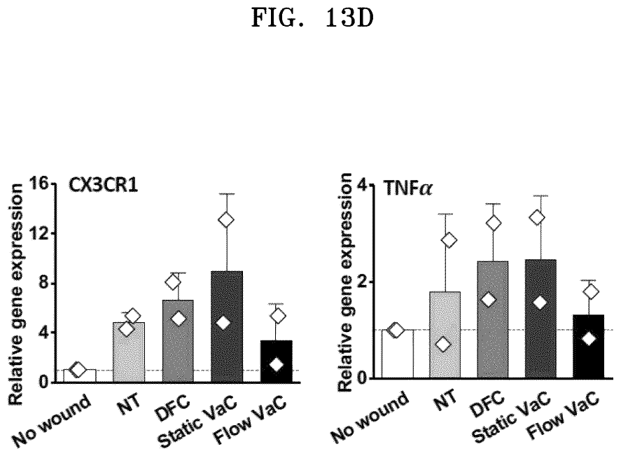

To confirm the therapeutic effect by the Flow Vac and specific effects of the related immune response, the distribution of macrophages recruited from the wound site was analyzed. iNOS, a surface marker of macrophages (M1) known to promote inflammatory responses, was expressed relatively highly in the DFC group, the Static Vac group, and the control group (FIG. 13A), whereas macrophages (M2), known to accelerate regeneration and wound healing, were observed highly in the Flow Vac group (FIG. 13B).

### 6-2. Confirmation of Expression of Chronic Wound-Related Genes

The expression levels of CD11b, IL-1β, CX3CR1, and TNF-α, which are genes known to be related to chronic wounds, were confirmed. As a result, the Flow Vac group showed significantly lower expression levels of these genes than the DFC group, the Static Vac group, and the control group (FIGS. 13C and 13D). Based on the results above, it was confirmed that the blood clots obtained by the microfluidic device according to an embodiment and by the method according to an embodiment could not only heal the wounds, but also inhibit the chronic progression of wounds by activating the immune system.

Referring to the overall results above, it is demonstrated that the microfluidic device according to an embodiment and the preparation method using the same could contribute to providing bioscaffolds or histocompatible transplant materials with enhanced functionality.

### Example 7. Culture and Differentiation of Muscle Cells by Using Microfluidic Device

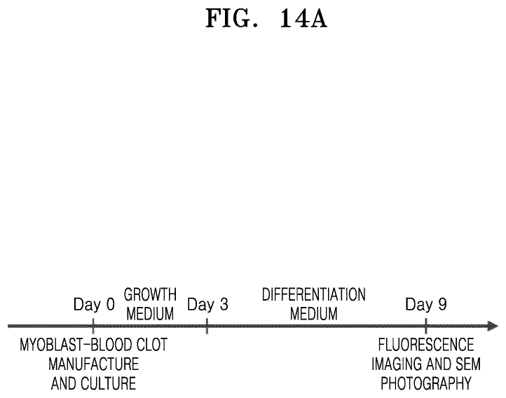

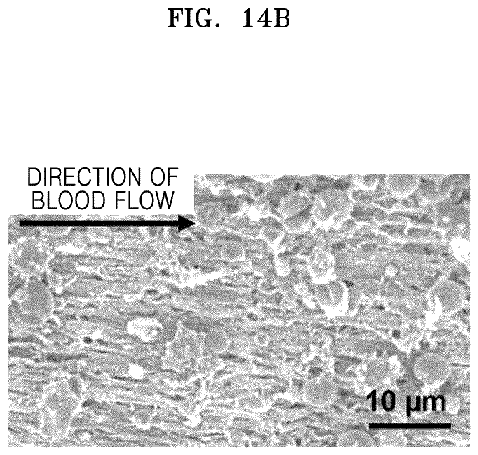

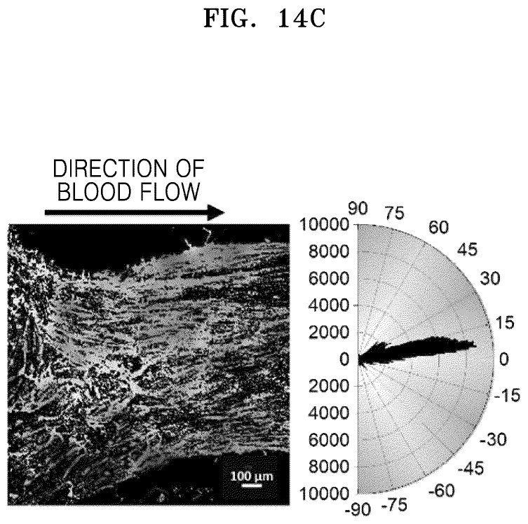

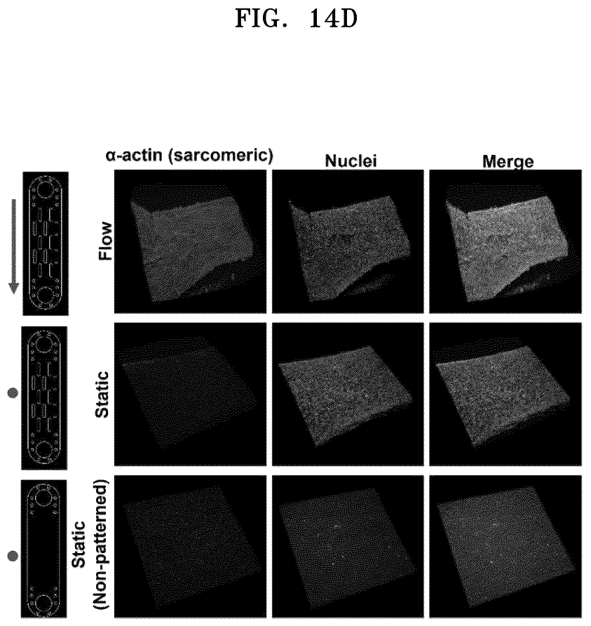

In this example, it was attempted to confirm the efficacy of controlling biological activity of cells in a bioscaffold prepared by the microfluidic device according to an embodiment. To this end, blood containing various cells and extracellular matrix components was used as biofluid, and this biofluid was mixed with muscle cells as target cells. Then, a cell culture was prepared in a similar manner to Example 2-1. A culture medium and a differentiation medium were each supplemented with blood clots containing myoblasts within the cover through the frame of the microfluidic device according to an embodiment. As shown in FIG. 14A, the cell culture was performed for a total of 12 days. Here, Dulbecco's modified Eagle's medium (DMEM) supplemented with 10% fetal bovine serum and 1% penicillin streptomycin was used as the culture medium, and DMEM supplemented with 2% horse serum and 1% penicillin streptomycin was used as the differentiation medium.

As a result, it was confirmed that the myoblasts and myofibers in the blood clots were aligned according to the direction of the blood flow (FIGS. 14B and 14C), and that the myoblasts highly expressed α-actin (sarcomeric) which is a myofiber differentiation marker (FIG. 14D). These experimental results above indicate that the proliferation and differentiation of myoblasts could be promoted through the cell cultures within the blood clots according to an embodiment.

That is, the microfluidic device according to an embodiment and the method of culturing cells by using the same can effectively control the growth, differentiation, function, and biological activity of cells.

1. A microfluidic device for manufacturing a bioscaffold, comprising:
a substrate for supporting a biofluid containing extracellular matrix components and a bioscaffold produced by the biofluid; and
a cover positioned on top of the substrate and configured to induce gelation of the biofluid through a flow of the biofluid,
wherein the cover includes one or more inlets through which the biofluid is injected, a microfluidic channel configured to induce shear stress to the injected biofluid, and one or more outlets capable of inducing a flow of the biofluid by discharging the biofluid from the microfluidic channel.

2. The microfluidic device of claim 1, wherein the extracellular matrix components are aligned according to a flow of the biofluid.

3. The microfluidic device of claim 1, wherein the substrate and the cover are separable from each other.

4. The microfluidic device of claim 1, wherein the shear stress of the biofluid capable of producing a bioscaffold from the flowing biofluid is 0.01 dyne/cm² to 10,000 dyne/cm².

5. The microfluidic device of claim 1, wherein the microfluidic channel further includes one or more micropillars capable of inducing shear stress, and
a height of the one or more micropillars is equal to or lower than a height of the microfluidic channel.

6. The microfluidic device of claim 5, wherein the height or interval of the one or more micropillars is 0.1 μm to 5 cm.

7. The microfluidic device of claim 5, wherein a cross section of the one or more micropillars is n-polygonal or amorphous, and n is 3 to 12.

8. The microfluidic device of claim 5, wherein a width of the one or more micropillars is 0.1 μm to 5 cm.

9. The microfluidic device of claim 5, wherein a length of the one or more micropillars is 0.1 μm to 5 cm.

10. The microfluidic device of claim 1, wherein the biofluid further contains at least one type of cells selected from the group consisting of vascular endothelial cells, muscle cells, stem cells, osteocytes, chondrocytes, cardiomyocytes, epidermal cells, fibroblasts, nerve cells, hepatocytes, enterocytes, gastric cells, skin cells, adipocytes, blood cells, immune cells, cell spheroids, and organoid cells.

11. The microfluidic device of claim 1, further comprising a frame seated on the cover and configured to provide an internal space for guiding a second biofluid containing a functional factor to the gelled biofluid.

12. The microfluidic device of claim 11, wherein a cross-sectional area of the frame is wider than a cross-sectional area of the cover.

13. A method of manufacturing a bioscaffold, comprising:
injecting a biofluid containing extracellular matrix components into the microfluidic device of claim 1; and
forming a bioscaffold from the injected biofluid.

14. The method of claim 13, wherein the extracellular matrix components are extracellular matrix proteins.

15. The method of claim 14, wherein the extracellular matrix proteins are aligned according to a flow of the biofluid.

16. The method of claim 15, wherein the extracellular matrix proteins aligned according to the flow of the biofluid include one or more selected from the group consisting of fibrin, collagen, fibronectin, von Willebrand factor, and laminin.

17. The method of claim 15, wherein the biofluid further contains at least one type of cells selected from the group consisting of vascular endothelial cells, muscle cells, stem cells, osteocytes, chondrocytes, cardiomyocytes, epidermal cells, fibroblasts, nerve cells, hepatocytes, enterocytes, gastric cells, skin cells, adipocytes, blood cells, immune cells, cell spheroids, and organoid cells.

18. The method of claim 15, wherein a flow velocity of the injected biofluid is 0.01 mL/hour to 1,000 mL/hour.

19. A method of culturing cells by using a bioscaffold, comprising:
injecting a first biofluid containing extracellular matrix components and cells into the microfluidic device of claim 1;
forming a bioscaffold from the injected biofluid;
separating the substrate of the microfluidic device and the cover on which the bioscaffold is formed from each other;
coupling the separated cover with a frame that is seated on the separated cover and provides an internal space for guiding a second biofluid containing a functional factor to the gelled biofluid; and
treating the frame seated on the cover with the second fluid containing the functional factor.

20. The method of claim 19, further comprising, after treating the frame with the second biofluid containing a functional factor, culturing the formed bioscaffold.

21. The method of claim 19, wherein the extracellular matrix components are extracellular matrix proteins.

22. The method of claim 21, wherein the extracellular matrix proteins are aligned according to a flow of the biofluid.

23. The method of claim 22, wherein the extracellular matrix proteins aligned according to the flow of the biofluid include one or more selected from the group consisting of fibrin, collagen, fibronectin, von Willebrand factor, and laminin.

24. The method of claim 19, wherein the cells are at least one type of cells selected from the group consisting of muscle cells, stem cells, osteocytes, chondrocytes, cardiomyocytes, epidermal cells, fibroblasts, nerve cells, hepatocytes, enterocytes, gastric cells, skin cells, adipocytes, blood cells, immune cells, cell spheroids, and organoid cells.

25. The method of claim 19, wherein the functional factor is a culture factor, a growth promoting factor, a differentiation inducing factor, or an expression inducing factor.

26. A composition for tissue transplantation, comprising a bioscaffold manufactured by the method of claim 13.
